(19) United States
(12) Patent Application Publication
(10) Pub. No.: US 2025/0049976 A1
(43) Pub. Date: Feb. 13, 2025

(54) Systems and Methods for Automated Garment Application and Defect Detection Along with Automated Garment Removal and Contaminant Analysis

(71) Applicant: Englover, LLC, Fayette, AL (US)

(72) Inventor: Van Clark Gravlee, Fayette, AL (US)

(21) Appl. No.: 18/708,554

(22) PCT Filed: Nov. 10, 2021
(86) PCT No.: PCT/US2021/058796

§ 371 Date: May 8, 2024

(51) Int. Cl. A61L 2/28; A61B 42/50

(52) U.S. Cl. CPC A61L 2/28; A61B 42/50; A61L 2202/26

(57) ABSTRACT

This technology may relate to systems and methods for donning, doffing, testing, containing, and tracking protective garments for use in environments where there is a high likelihood of hazardous contamination to the user, patient, pharmaceutical culture, or the surrounding area or use in any other application where a protective garment is used. The technology disclosed may utilize systems and methods to non-destructively detect pinholes, other defects and variabilities, or the like, in elastomeric gloves at multiple points, including but not limited to the point of manufacture and immediately pre-donning of the glove. The technology disclosed may also relate to removing elastomeric garments, such as gloves, boots, prophylactic devices, in a manner that may prevent the spread of contaminants while preserving the integrity of those contaminants for analysis. It may also analyze those contaminants and place the results of that analysis in databases for contact tracing and other infection control measures.

### TECHNICAL FIELD

This patent generally relates to the field of donning, doffing, testing, containing, and tracking protective garments used in areas having possible pathogen, bacterial, viral, or other hazardous contamination risks, and apparatus and methods of using, manufacturing, and packaging the same. More specifically, this patent may relate to systems and methods for donning, doffing, and containing protective gloves for use in situations where there may be a likelihood of harmful or hazardous contamination either to or from the garment user in contact with a patient, a culture, or the surrounding environment or any other application where a protective garment is used. The technology disclosed may utilize systems and methods to non-destructively detect pinholes, other defects and variabilities, or the like, in elastomeric gloves at multiple points, including but not limited to the point of manufacture and immediately pre-donning of the glove. The technology disclosed herein may also relate to removing elastomeric garments, specifically gloves, but also other exemplary garments such as gloves, boots, hoods, prophylactic devices, in a manner that may prevent the spread of contaminants while preserving the integrity of those contaminants for analysis. It may also analyze those contaminants and place the results of that analysis in databases for contact tracing and other infection control measures.

### BACKGROUND

From the COVID-19 pandemic and other disease outbreaks, such as Ebola, the world as a community has come to realize the importance of its medical and first responders to be properly protected and to properly contain any pathogen or hazard so they do not spread. One possible risk mitigation measure that these individuals may take is to wear protective elastomeric gloves. Donning and doffing elastomeric gloves may be accomplished by several different conventional methods and apparatus. However, while these methods and apparatus may have been used for many years, substantial problems with respect to real-time defect detection and post-use disposal, and the like, remain unresolved. U.S. Pat. No. 9,925,015 may provide a wearable protective article donning system in an automated or semi-automated donning system that may be used to mechanically don wearable protective articles, such as gloves, without human contact with the outer surface of the wearable article. Thus, the '015 system may be used to render wearable protective articles or garments to be less capable of transferring disease organisms than a manually donned protective article or garment. In the '015 donning system, a vacuum device has the ability to prepare an article for use in the donning system by inflating the article by creating a pressure differential between the articles inner and outer surface, where atmospheric pressure is present on the inner surface of the article and a negative pressure, or vacuum is applied to the outer surface. Packaging of the wearable protective article may maintain sterility of the article and allow preparation of the article for donning without human contact. However, there may not be an adequate way to check the article or garment for possible defects, such as but not limited to pinholes. In addition, wearable protective articles used in the system may be subject to removal techniques and post-use treatments that may destroy the evidence chain of pathogens, making contact tracing, epidemiology, and mitigation extremely difficult. Alternatively, or in addition, a body part of the user may be subject to treatments that may destroy potential pathogens during insertion into a wearable protective article prepared for donning by the system. However, the system of the '015 patent fails to provide an adequate solution to detect and alert the user of possible defects in the garment that are present prior to donning. It additionally fails to provide an adequately safe way for the garment to be removed, contained, and disposed of. These shortcomings leave much to be desired for systems and methods of donning and doffing protective garments. The technology disclosed herein may allow for a more comprehensive, user-friendly experience by utilizing improved contamination containment and defect detection systems and methods.

The Covid-19 pandemic has exposed potential weaknesses in the testing for spreading pathogens and other contaminants. One potential weakness is the absence of a uniform early detection system that identifies areas of concern before they become large regions of infection. Another weakness in current practice is the lack of randomized testing that quantifies the levels and locations of infection in statistically valid ways. Both of these major testing failures have hampered epidemiology and infection control efforts. Unreliable data and the lack of data continually keep testing and containment efforts from hitting the right targets at the right time to effectively and efficiently slow the progression of covid and other pathogens. The system disclosed herein may improve on even the best current practice. In some embodiments testing may occur both granularly by a single garment and in bulk by a group of garments. In some embodiments, reporting of positive findings can be reported as they are detected via an encrypted internet connection. For the world's future epidemics, a fast, directed, and pinpointed response may be beneficial to assist in containing an outbreak and preventing an epidemic from evolving into a pandemic.

Doffing of gloves and other elastomeric garments may often be a manual rather than an automated process. However, manual rather than automated removal of gloves and other elastomeric garments may create significant personal and environmental contamination issues. Negative pressure doffing systems such as described in U.S. Pat. Nos. 4,915,272 and 5,058,785 are available but may present significant risks with vacuum chamber contamination and loss of garment contaminant identification during the removal process. In institutions, collection and testing of contaminants may be most commonly done with swabs and may be a time consuming and inefficient process that may have a high likelihood of exposing another individual to those contaminants. It may be important to import the contamination collection data into epidemiology or forensics databases; however, when this is done manually using swab results, there may be instances of mis-entry of data and large inefficiencies that may create an increased risk of new contaminations and outbreaks due to lack of tracking. In one embodiment of the application, the technology aims to automatically contain and track contaminants as part of the removal of the protective garment in a way that isolates the contaminants from the removal system.

Another aspect of the protective garment market and specifically protective gloves, may be the inefficiency of quality control. Quality control may often be done off the manufacturing line and away from the point of glove installation, possibly in destructive lab-based procedures that may only test a small sample size of the manufactured gloves. Whether these procedures are done at the manufacturing facility or by an independent certification lab, they may generally be done on a random sample that may be meant to statistically represent the entire batch or run of gloves manufactured at that time. The FDA has standards for patient examination gloves (ASTM D3578), medical-surgical gloves (ASTM D3577), and PVC gloves (ASTM D5250).

A critical test that can be done on gloves is the “watertight” test (ASTM D5151) which checks gloves for pinholes. An example of this test may involve suspending each glove and filling it with 1000 milliliters of room temperature water. The glove may then be examined for visual leaks immediately after the addition of the water and then two minutes after the addition of the water. If no water escapes from the glove, it passes the test.

Gloves may be sold as meeting an Acceptable Quality Level (AQL). To determine the AQL of gloves, if a manufacturer produces, for example, 10,000 gloves from the same material and uses the same settings and processes, perhaps 200 gloves will be randomly pulled from the production line. In order to meet a common medical glove designation of a 2.5 AQL, no more than five gloves can fail the quality test. If more than five gloves fail the test, then the entire batch may effectively fail and therefore may be discarded or perhaps sold as utility gloves unless each glove is tested individually for quality. AQLs of 3.4 and 1.5 may also be common designations for medical gloves.

The ability to identify specific bacteria, viruses, individual humans, users, and any other organism, as well as specific genes such as those that code for resistance to antibiotics, may have become much more powerful and far less expensive in the past few years; this trend may likely continue. Advances in microfluidics and PCR (polymerase chain reaction) may have made testing both quick and inexpensive. Immunoassay techniques may also be advancing and may be used to test for many nongenetic contaminants and toxins. Beyond these methods, one may employ many varieties of spectroscopy, chemical indicators, and even biological culturing.

Gloves and boots may be ideal devices with which to sample surfaces and objects. They may naturally collect the substances and lifeforms that may be of concern. They may be manufactured or treated to collect and analyze even more effectively. The microbial flora present in a room and from the skin, clothing, and exhalate of the room's occupants may be broadly represented by the viable and nonviable microbes and microbe structures which settle on surfaces in the room, such as the floor, table tops, and the like.

### DISCLOSURE OF INVENTION

In general, the technology described herein may involve both devices and methods in a variety of embodiments to achieve efficient and safe donning, doffing, containment, manufacturing, and testing of protective gloves for use in environments where there is a high likelihood of hazardous contamination to the user or the surrounding area, or use in any other application where a protective glove or other prophylactic item is used. In one embodiment of the technology described herein, each component may have multiple sizes and style options, each varying embodiment may stem from essentially the same design, which may be an automated or semi-automated donning and doffing system and methods, contamination tracking systems, and methods defect detection and correction system and methods, and contamination and material testing systems and methods. In addition, the technology described herein may be captured in different embodiments, including but not limited to a defect and contamination detection and containment doffing and donning machine or become systems, methods, apparatus, or processes, or the like.

The technology described herein may be but is not limited to a system and method for testing gloves in real-time, possibly at the point of manufacture or the point of donning. Rather than testing for leakage of water (or of air), as may typically be done in a manufacturing setting with a sample of gloves, the glove may be slightly inflated once automatically stripped from a form and illuminated from the inside with a light or a scanning laser, or the like. Inflation may occur through traditional means or through the use of a negative pressure vacuum chamber, such as is well known to those skilled in the art and also by applying details as available in the references, all hereby incorporated by reference. This may be useful in detecting even tiny pinhole defects, which may project a bright point of light from that pinhole. Cameras may be located externally of the garment that may detect those relatively brighter, more visible escapes of light. Alternatively, cameras may be located within the inflated glove and near the source of light or a laser or other scan which may detect the bright light or non-reflection of light evidenced by the presence of a pinhole. All such methods may also help reveal other glove defects, such as variations in thickness and thin or weak areas which show up as differences in opacity or translucency.

In some embodiments, a computer vision algorithm may determine in real-time, or close to real-time (such including in seconds, a minute, or coordinated to be accomplished during the specific activity, operation, or manufacturing action involved), a type of defect and may immediately dispose of a rejected glove or reroute it to be used for a non-medical purpose. The presence of certain defects may also provide electronically controlled or alarm-based feedback to the manufacturing line, allowing adjustments to be made to varying processes, including but not limited to dipping, curing, or formulations of rubber or coagulants of the garment. Such adjustments may also be automatically implemented.

In some embodiments, gloves may be packaged or repackaged in a process post manufacture. In some embodiments this process may or may not be a part of the manufacturing line. It may be beneficial for the garment, such as a glove, to be manually or automatically placed on a fixture that allows for the glove to be inflated, checked for defects, sterilized with UVC, a spray or a combination of the two, serialized with a barcode or embedded RFID, oriented in a pre-determined fashion (e.g., thumbs to the left) and packaged in boxes, other containers, or the like.

In some embodiments, these detection methods may also be used immediately prior to donning as a component of a glove donning machine, such as but not limited to the “Englover Donning System.” In this usage, while a glove is inflated, the light source may be in a cover that goes over the open wrist of an inflated glove. In one embodiment, the computer vision system cameras may be located within the vacuum chamber of the glove donning machine to detect escaped or pass-through light or within the same cover to detect non-reflected light. In some embodiments, a sophisticated stress analysis of a glove may be done with the computer vision system perhaps even as its inflation is varied. The computer vision system utilizing machine learning or artificial intelligence may correlate the relative inflation of different parts of the glove with the structural strength of the glove and its parts.

In some embodiments, including but not limited to both the manufacturing and pre-donning embodiments, the use of a biocidal light source such as UVC or far UVC may sterilize the glove. Certain glove formulations such as those with titanium dioxide (TiO2) may enhance the effectiveness of UVC and far UVC through photo activation. In some embodiments, TiO2 may be used in glove manufacture or other UV activated metal salts such as, but not limited to, AgNO3 may also be applied to the glove during a sterilization process or may be added to the material formulation of the glove during manufacture.

### BRIEF DESCRIPTION OF DRAWINGS

FIG. 1 is an exemplary elastomeric, protective garment pre-containment test system in accordance with some embodiments of the technology described herein.

FIG. 2 is another exemplary elastomeric, protective garment pre-containment test system illustrating garment removal and containment in accordance with some embodiments of the technology described herein.

FIG. 3 is an exemplary elastomeric, protective garment tracking system in accordance with some embodiments of the technology described herein.

FIG. 4 is an exemplary computer-controlled elastomeric, protective garment defect detection system in accordance with some embodiments of the technology described herein.

FIG. 5 is an exemplary elastomeric, protective garment light transmission defect detection system in accordance with some embodiments of the technology described herein.

FIG. 6 is an exemplary illustration of a method of elastomeric, protective garment pre-containment testing in accordance with some embodiments of the technology described herein.

FIG. 7 is an exemplary illustration of a method of elastomeric, protective garment tracking in accordance with some embodiments of the technology described herein.

FIG. 8 is an exemplary illustration of a method of computer-controlled elastomeric, protective garment defect detection in accordance with some embodiments of the technology described herein.

FIG. 9 is an exemplary illustration of a method of elastomeric, protective garment light transmission defect detection in accordance with some embodiments of the technology described herein.

FIG. 10 is an exemplary elastomeric, protective garment pre-containment testing system further illustrating a threshold contaminant detection system data storage system configured to connect to a cloud computing network.

FIG. 11 is an exemplary elastomeric, protective garment tracking system illustrating one embodiment connected to a cloud computing network.

FIG. 12 is an exemplary elastomeric, protective garment pre-containment testing system network configured to connect to a threshold contaminant detection system data storage system configured to connect to a cloud computing network.

FIG. 13 is an exemplary illustration of a method of elastomeric, protective garment repackaging in accordance with some embodiments of the technology described herein.

### MODES FOR CARRYING OUT THE INVENTION

It should be understood that the technology includes a variety of aspects, which may be combined in different ways. The following descriptions are provided to list elements and describe some of the embodiments of the application. These elements are listed with initial embodiments; however, it should be understood that they may be combined in any manner and in any number to create additional embodiments. The variously described examples and preferred embodiments should not be construed to limit the embodiments of the application to only the explicitly described systems, techniques, and applications. The specific embodiment or embodiments shown are examples only. The specification should be understood and is intended as supporting broad claims as well as each embodiment and even claims where other embodiments may be excluded. Importantly, disclosure of merely exemplary embodiments is not meant to limit the breadth of other more encompassing claims that may be made where such may be only one of several methods or embodiments which could be employed in a broader claim or the like. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

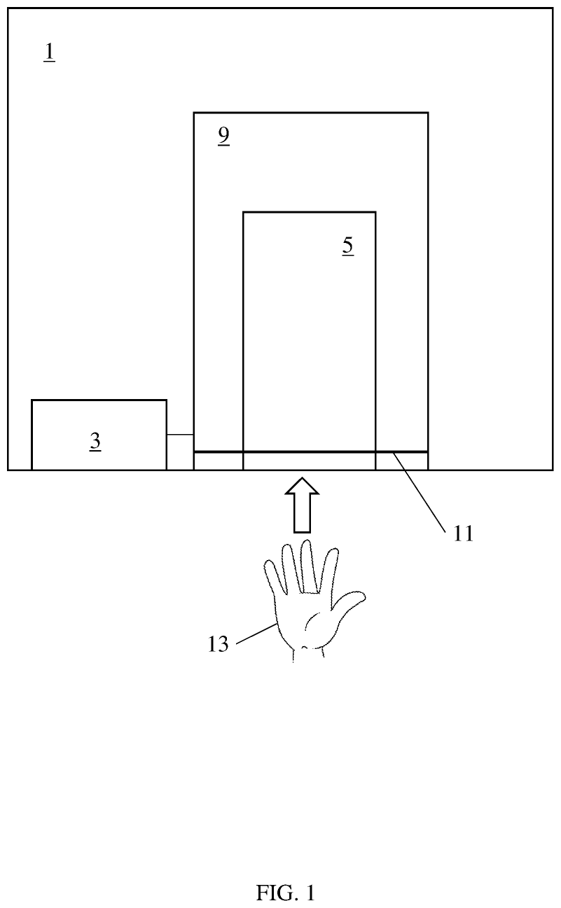

FIG. 1 is an exemplary embodiment of an elastomeric, protective garment pre-containment test system. In some embodiments, an elastomeric, protective garment pre-containment testing system (1) may be utilized. In some embodiments, this system may include but is not limited to a garment doffing system (9), a threshold contaminant testing system (11), a contaminant notification control (3), and a containment system (5). In some embodiments, a garment doffing system may be a mechanical garment doffing system, a negative pressure garment doffing system, or the like as is known in the art. In some embodiments, a mechanical garment doffing system may utilize, for example, hooks, claws, prying arms, expanding claws, and the like to assist the user in no user contact removal of the garment. In other embodiments, it may be beneficial to utilize a negative pressure garment doffing system. In some embodiments, a negative pressure garment doffing system may be a system that creates a pressure differential between the inside of a garment and the outside. This pressure differential may be achieved by creating a vacuum in a sealed chamber that may have an opening for the garment to go in with a seal, either mechanical, pressure-related, adhesive related, or the like, and then utilizing atmospheric pressure on the inner, user body contact, side of the garment to inflate the garment.

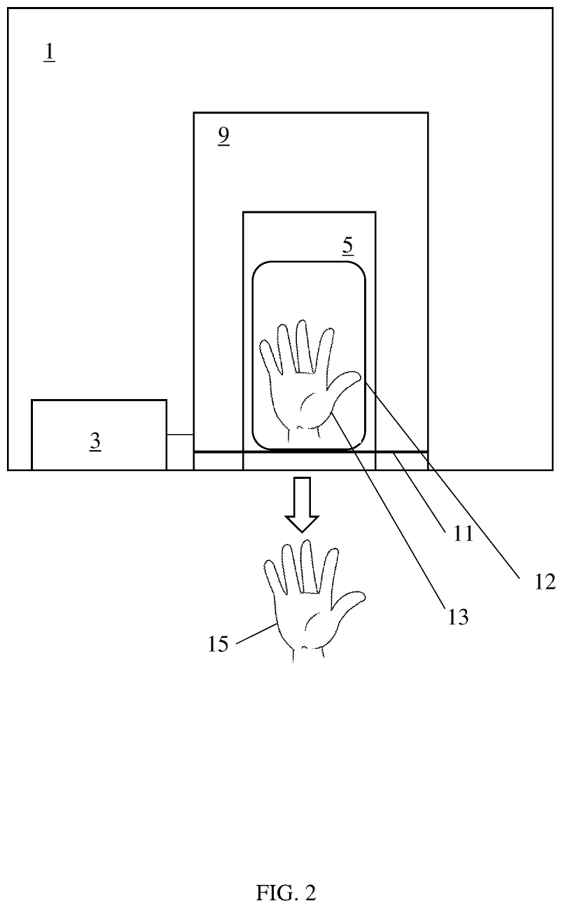

In some embodiments, such as shown in FIG. 2, a threshold contaminant detection system for contaminated garments (13) may include but is not limited to a contaminant scanner, a detection plane that, for example, a user's gloved hand passes through entering a containment zone, or the like. In some embodiments, a contaminant scanner may utilize different spectrums of light to scan for pathogens, bacteria, viruses, or the like. The contaminant scanner may be of any other scanning technology such as but not limited to fluorescence, immunofluorescence, or the like. In some embodiments, the threshold contaminant detection system may be but is not limited to a detection plane, or the like. In some embodiments, the threshold contaminant detection system may wash, rinse, extract, or the like a sample from a garment and perform detection using rapid PCR testing, immunology-based methods, DNA analysis, or the like. In some embodiments, a contamination notification control may be present that may be configured to communicate with a containment system. A contamination notification control may be an automatic function that may be controlled by a computer-controlled function to contain and isolate a contaminated garment. In other embodiments, a contamination notification control may be a visual, audible, sensory, or the like, signal to a user to notify the user of a contaminant present. A sensory signal may be but is not limited to a vibration. In further embodiments, the pre-contaminant test system may include but is not limited to a containment system responsive to a contaminant notification control. The contaminant notification control may be computer controlled to alert the containment system of a contaminant and may trigger the containment system to automatically contain the contaminated garment for further testing as the garment is removed from a user's body. In another embodiment, the containment system may be a manual process that, based on an indication from the contaminant notification control, may require input, such as but not limited to a switch, button, voice command, touchless command, so a user's hand (15) may be free of contact, or the like to contain the contaminated garment.

FIG. 2 is an exemplary embodiment of a pre-containment test system illustrating garment removal and containment. As shown in FIG. 2, in some embodiments, the containment system (5) may be but is not limited to an isolated compartment positioned inside the garment doffing system configured to protect other components of the garment doffing system and the pre-containment testing system from contamination. It may also be beneficial in some embodiments to utilize a containment system that is removable from the pre-containment testing system, such as but not limited to a separable sealable bag (12), a sterile garment that is free from contaminants to contain the contaminated garment, or another container type for further testing, disposal, or the like. In other embodiments, it may be beneficial to combine the vacuum chamber of a negative pressure doffing system with a containment system to create a sterile containment vacuum chamber that is a vacuum chamber that is free of contaminants with the ability to removably contain a contaminated garment.

In some embodiments, it may be beneficial to use testing methods that may provide immediate or near immediate testing results using varying spectroscopy techniques. This may be accomplished by utilizing varying wavelengths of light because some bacteria may fluoresce or absorb certain wavelengths of visible and nonvisible light. One possible advantage that this method may present is that there may be zero contact from the testing system to the contaminants on the garment, thereby protecting the contaminants for future testing or disposal. In another embodiment it may be beneficial to utilize rapid antibody testing that may produce results near instantaneously for certain bacteria or viruses. This may be accomplished by an automated swabbing, scraping, or removal of a sample in a like manner from the garment and testing. PCR (polymerase chain reaction) testing may improve as cycle times are reduced and less duplicated genetic material is needed to perform tests.

In some embodiments it may be beneficial to wash a contaminated garment with a solution that may denature the viral or bacterial load on the garment to assist in safer disposal of the garment. In some embodiments, it may be beneficial to contain the contaminated solution that is washed off from the garment to test for specific contaminants. In some embodiments it may be beneficial to contain a portion of the contaminated waste from each garment in a containment system such as a sealed bag, capillary, other tube, or the like and to mix the remaining wash into a containment system for a number of garments, such as those from an entire surgical team on a procedure, a particular shift in a single ward or ICU unit, a specific housekeeping group, or the like to recognize larger contamination. In some embodiments testing a larger batch of garments may assist in narrowing testing to specific individual garment containment systems to more efficiently and effectively test, focusing then on the contaminants found in the larger batch.

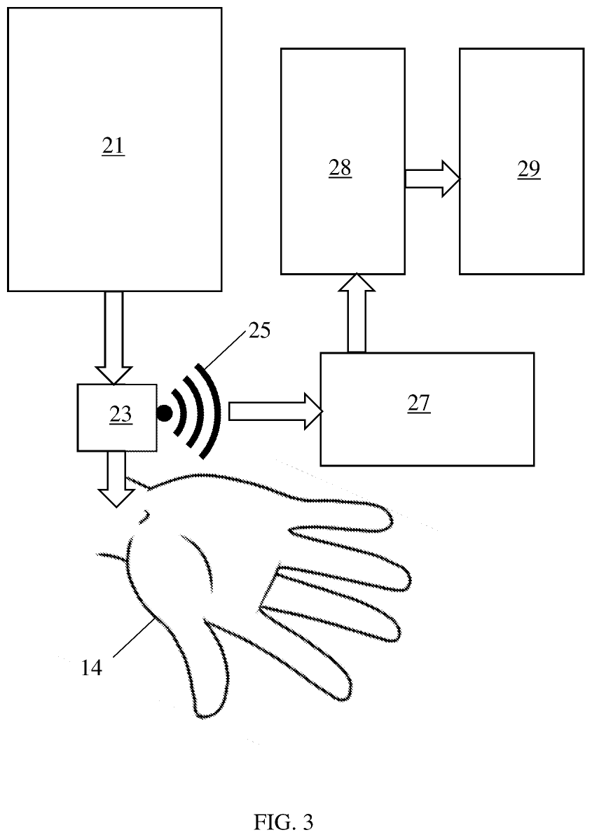

FIG. 3 is an exemplary embodiment of an elastomeric, protective garment tracking system in accordance with the technology disclosed herein. In some embodiments, an elastomeric, protective garment tracking system may be utilized. In some embodiments, this system may include but is not limited to a tracking tag embedding system (21), a remote tracking tag (23), a remote tracking tag output (25), a receiving system (27), a data storage system (28), and a contaminant locator (29). In some embodiments, the tracking tag embedding system may be but is not limited to a user applying a locator tag to each garment; this process may also be automated. The tracking tag embedding system may also be done during the manufacturing or donning phase of the garment through the use of automated controls, such as but not limited to roll on, mold in, or printed application. In some embodiments, a remote tracking tag that may be applied by a track tag embedding system may be used to track the location of the garment and its uses. A remote tracking tag may be but is not limited to an RFID tag, a printed barcode, a molded barcode, a chemical tag, an electromagnetic tag, a radiological tag, a Bluetooth transmitter, NFC transmitter, LoRa transmitter, wireless transmitter, or the like that may provide an output, be read by a sensor, or the like. Said output may be a physical transmission, broadcast, or electronic transfer of data or the ability to be recognized by a receiving system, or otherwise. Some embodiments may include a receiving system that may be configured to receive a remote tracking tag output. The receiving system may be but is not limited to an optical sensor, chemical sensor, electromagnetic sensor, radiological sensor, RFID scanner, barcode scanner, Bluetooth receiver, wireless receiver, NFC receiver, LoRa receiver, or the like. The receiving system may receive data such as but not limited to the time of garment donning, duration of garment usage, and location of usage. In some embodiments, it may be beneficial to utilize a data storage system to store the data that is received by the receiving system. In some embodiments, a data storage system may be capable of storing computer-readable media, may be but is not limited to a computer, random access memory, hard disk, cloud storage, or the like. In some embodiments, it may be beneficial to store data for further processing and to locate the source of a contaminant or the failure mechanism, such as the time the garment was worn to further improve safety for the user and the environments in which the garments are worn. In some embodiments, a contaminant locator (29) may be utilized. This may be a scan upon entering a room or performing a different task where the remote tracking tag is recorded, and if there is a contaminant present, then the contaminant locator (29) may add a tag, such as but not limited to a location, time of contact, time of scan, or contaminant type tag to the computer-readable media corresponding to the specific garment remote-tracking tag. A new task may be but is not limited to switching from one workstation to the next or from patient to patient.

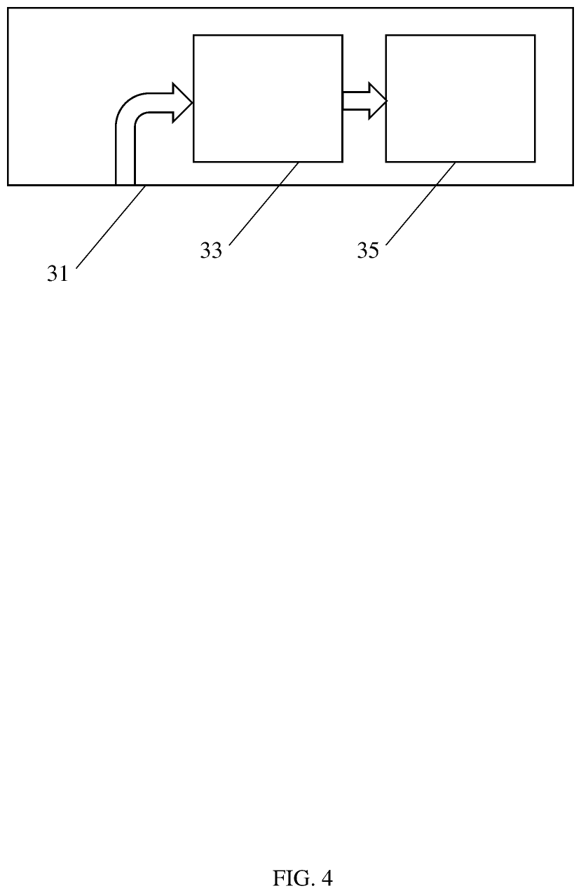

FIG. 4 is an exemplary embodiment of a computer-controlled elastomeric, protective garment defect detection system, in accordance with the technology disclosed herein. In some embodiments, a computer-controlled elastomeric, protective garment defect detection system may be utilized to more rapidly detect garment defects. In some embodiments, the computer-controlled elastomeric, protective garment defect detection system may include but is not limited to a detection system configured to detect defects in garments (31), a trigger (33), an accept-or-reject sorting function (35). In some embodiments, the detection system may be configured to detect defects in garments, and may be a computer-controlled vision system that may include but is not limited to a photo sensor array, a camera, or the like that detects differences in light transmission through the garment to detect a defect, such as but not limited to thickness variabilities, surface imperfections, surface cuts, surface divots, or the like. In another embodiment, it may also be an elastomeric, protective garment light transmission defect detection system as described herein. This system may occur at the point of manufacture, in inline or offline packaging, or at the point of donning the garment. The system may also use other computer-controlled vision systems, including but not limited to X-ray, machine vision, and the like. The computer-controlled vision system may also include a computer vision algorithm that may determine in real-time, or close to real-time (such including in seconds, a minute, or coordinated to be accomplished during the specific activity, operation, or manufacturing action involved) the type of defect and may immediately dispose of a rejected glove or reroute it to be used for a non-medical purpose. In some embodiments, this may be accomplished by utilizing a trigger, wherein if the detection system detects a defect, then the trigger may be triggered. In some embodiments triggering the trigger may effect an accept-or-reject sorting function or algorithm to accept or reject the detected garment based on whether or not that garment is suitable for a specific predetermined purpose. An accept-or-reject sorting function may be preprogrammed with acceptable limits for specific uses such as medical, general, clean room, or the like. These may be based on standards set forth from agencies such as but not limited to the FDA or the ASTM.

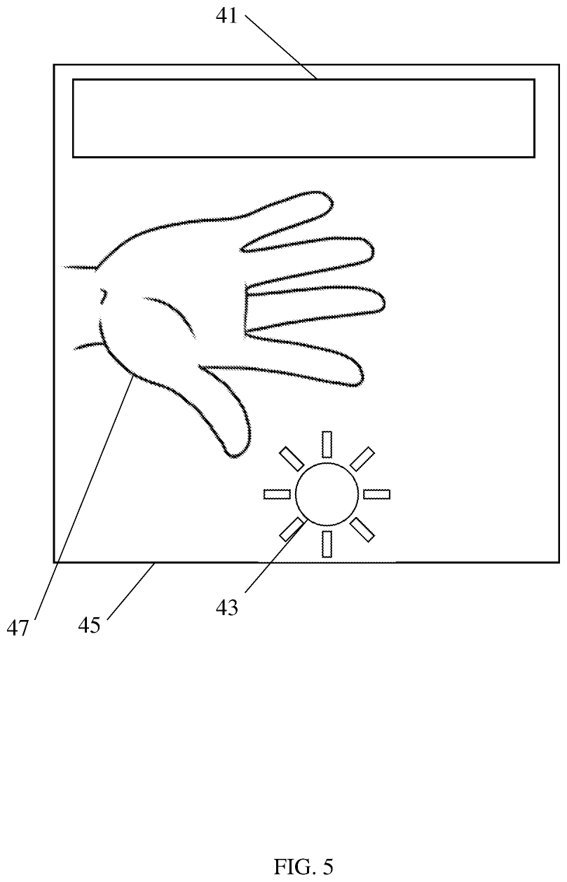

FIG. 5 is an exemplary embodiment of an elastomeric, protective garment light transmission defect detection system, in accordance with embodiments of the technology disclosed herein. Similarly, in some embodiments, an elastomeric, protective garment light transmission defect detection system may be utilized to detect defects and improve user safety before donning a garment. The elastomeric, protective garment light transmission defect detection system may include but is not limited to a light detection device (41), a light source (43), a negative pressure garment inflation chamber (45), and an inflated garment (47). A light detection device may include but is not limited to a garment external camera, a garment internal camera, a photosensor array, a reflected light detector, a pass-through light detector, or the like. The sensors may utilize a variety of sensors in combination or alone, such as a photoresistor, photo capacitor, LED sensor, or the like. In some embodiments, a light source may be but is not limited to at least one light-emitting diode or at least one laser, an LED or laser array may also be used. In some embodiments, a negative pressure garment inflation chamber may be used to inflate a garment by sealing the garment and then pulling a vacuum on the inside of the chamber. By pulling a vacuum, the pressure on the inside of the garment may be higher than the pressure in the inflation chamber, which may provide the sensation that the garment is inflated. In some embodiments, once the garment is inflated, the light source may be configured to shine light from a first end of the negative pressure inflation chamber to a second end where the light detection device is positioned. By shining light through an inflated garment, a light detection device may be able to measure the light transmitted through an inflated garment. In some embodiments, the first end may be a garment opening end where a user may place, for example, his hand through the garment opening end to assist in the removal of the garment. In other embodiments the second end may be the garment opening end. In some embodiments, the light detection device may be able to detect variabilities in the garment's material thickness and relay that information to the computer vision system mentioned herein to determine the material strength of the garment. This may be accomplished by applying known image recognition or image artificial intelligence capabilities that may even compare images or garments and may be applied to identify anomalies or statistically excessive variations In one embodiment, the glove may be slightly inflated, possibly by using traditional inflation techniques, such as but not limited to a balloon-type inflation system, once automatically stripped from a form and illuminated from the inside with a light or a scanning laser. This may be useful in detecting even tiny pinhole defects, which may project a variation such as a bright point of light from that pinhole. Cameras may be located externally that may detect those brighter escapes of light. Alternatively, cameras may be located within the inflated glove and near the source of light or a laser scan which may detect the bright light or non-reflection of light evidenced by the presence of a pinhole. In some embodiments, movable light sources, mirrors, and/or light detection devices can also be used. All methods may help reveal other glove defects, such as variations in thickness and thin or weak areas which may show up as differences in opacity or translucency.

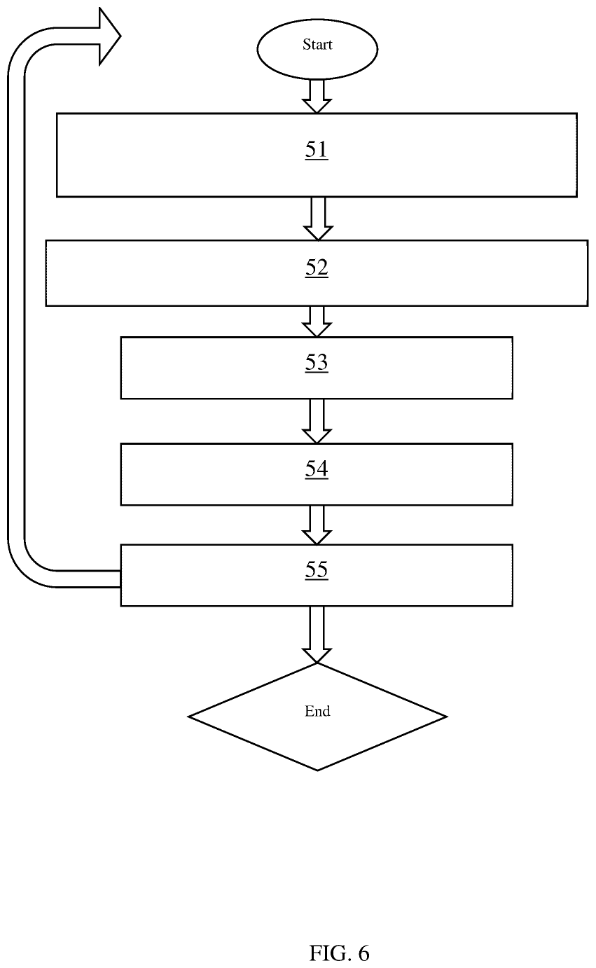

FIG. 6 is an exemplary illustration of a method of elastomeric, protective garment pre-containment testing in accordance with some embodiments of the technology described herein. In some embodiments, a method of elastomeric, protective garment pre-containment testing may include the steps of, but is not limited to the steps of, removing a garment automatically from a user (51), testing said garment for contaminants (52), automatically detecting contaminants (53), triggering a response for detected contaminants (54), and automatically containing a contaminated garment (55). In some embodiments, removing a garment automatically from a user may be done by a garment doffing system, a negative pressure garment doffing system, or the like. In some embodiments, testing the garment for contaminants may be accomplished by passing the garment through a threshold contaminant testing system. In some embodiments, triggering a response for detected contaminants may be utilizing a contamination notification control to determine what to do with or in the event of a contaminated garment. In some embodiments, it may not be necessary to contain the garment or hold the garment for further testing depending on the contaminant detected. Automatic decisions, indications, instructions, or the like may be made based on the application involved. In some embodiments, if the contaminant is one of particular importance, then automatically containing a contaminated garment (13) may be beneficial. In some embodiments, it may be beneficial to contain the contaminated garment in a containment system (5) for further test, safe disposal, or the like.

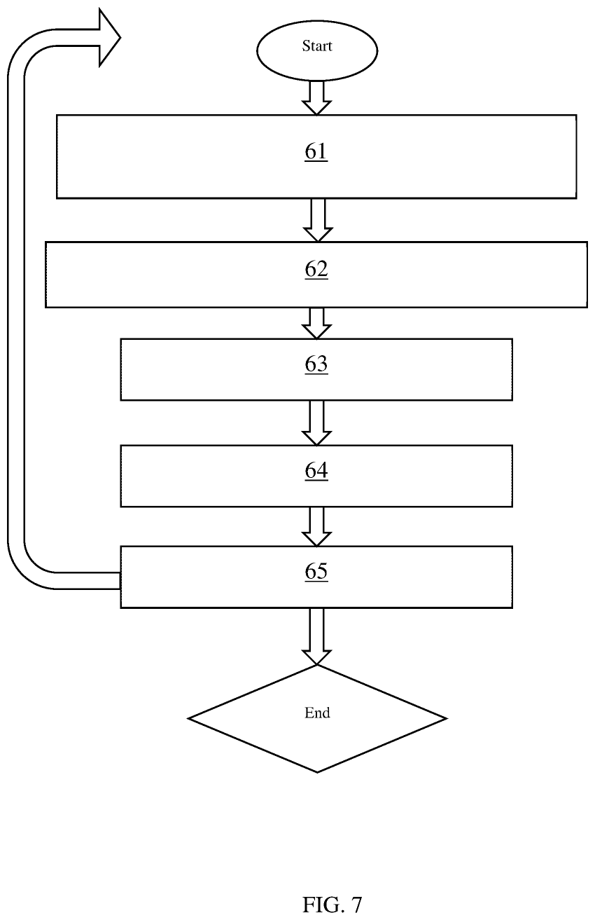

FIG. 7 is an exemplary illustration of a method of elastomeric, protective garment tracking in accordance with some embodiments of the technology described herein. In some embodiments, a method of elastomeric, protective garment pre-containment testing may include the steps of but is not limited to the steps of embedding a remote tracking tag to a garment (61), receiving an output from the remote tracking tag to a receiving system (62), storing data input into the receiving system in a data storage system (63), analyzing the data (64), and tracking the point of contamination based on analyzed data (65). In some embodiments, the tracking tag embedding system may be but is not limited to a user applying a locator tag to each garment; this process may also be automated. The tracking tag embedding system may also be done during the manufacturing or donning phase of the garment through the use of automated controls, such as but not limited to roll on, mold in, or printed application. In some embodiments, a remote tracking tag that may be applied by a track tag embedding system may be used to track the location of the garment and its uses. A remote tracking tag may be but is not limited to an RFID tag, a printed barcode, a molded barcode, a chemical tag, an electromagnetic tag, a radiological tag, Bluetooth transmitter, NFC transmitter, LoRa transmitter, wireless transmitter, or the like that may provide an output. Said output may be a physical transmission or electronic sending of data or the ability to be recognized by a receiving system, or otherwise. Some embodiments may include a receiving system that may be configured to receive a remote tracking tag output. The receiving system may be but is not limited to an optical sensor, chemical sensor, electromagnetic sensor, radiological sensor, RFID scanner, barcode scanner, Bluetooth receiver, wireless receiver, NFC receiver, LoRa receiver, or the like. The receiving system may receive data such as but not limited to the time of garment donning, duration of garment usage, and location of usage. In some embodiments, it may be beneficial to utilize a data storage system to store the data that is received by the receiving system. In some embodiments, a data storage system capable of storing computer-readable media may be but is not limited to a computer, random access memory, hard disk, cloud storage, or the like. In some embodiments, it may be beneficial to store data for further processing and to locate the source of a contaminant or the failure mechanism, such as the time the garment was worn to further improve safety for the user and the environments in which the garments are worn. Elements may be synchronized with users or actively inform a system or used as well. In some embodiments, a contaminant locator may be utilized. This may be a scan upon entering a room or performing a different task where the remote tracking tag is recorded, and if there is a contaminant present, then the contaminant locator may add a tag, such as but not limited to a location, time of contact, time of scan, or contaminant type tag to the computer-readable media corresponding to the specific garment remote-tracking tag. A new task may be but is not limited to switching from one workstation to the next or from patient to patient and thus it may be easier and more efficient to track, verify, or the like particular safety, sanitation, or the like procedural compliance.

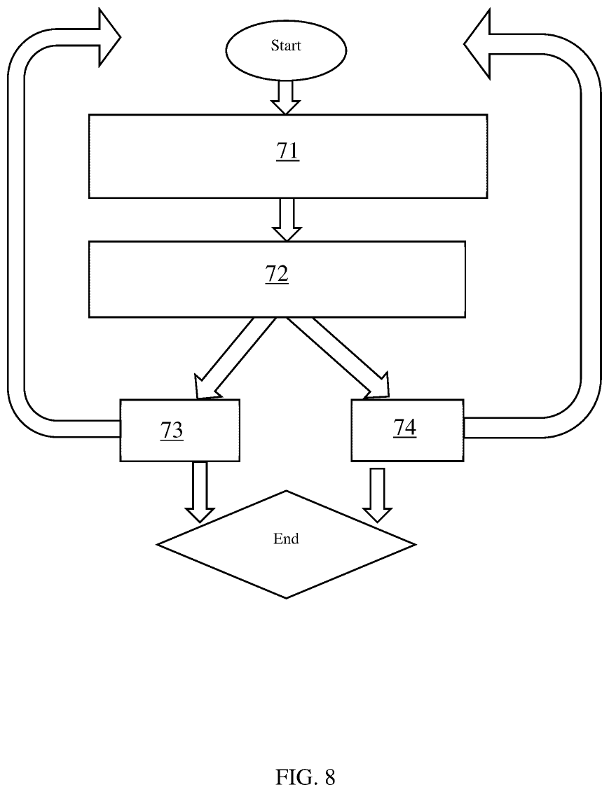

FIG. 8 is an exemplary illustration of a method of computer-controlled elastomeric, protective garment defect detection in accordance with some embodiments of the technology described herein. In some embodiments, a method of elastomeric, protective garment pre-containment testing may include the steps of but is not limited to the steps of detecting defects in garments (71), triggering when the defects in garments are detected (72), accepting garments that meet a predetermined quality threshold (73), and rejecting garments that fail to meet a predetermined quality threshold (74). In some embodiments, the detection system configured to detect defects in garments may be a computer-controlled vision system that may include but is not limited to a photo sensor array, a camera, AI systems, or the like that detects differences in light transmission through the garment to detect a defect, such as but not limited to thickness variabilities, surface imperfections, surface cuts, surface divots, or the like. In another embodiment, it may also be an elastomeric, protective garment light transmission defect detection system as described herein. This system may occur at the point of manufacture or at the point of donning the garment. The system may also use other computer-controlled vision systems, including but not limited to X-ray, machine vision, and the like. The computer-controlled vision system may also include a computer vision algorithm or an AI (artificial intelligence) system that may determine in real-time, or close to real-time (such including in seconds, a minute, or coordinated to be accomplished during the specific activity, operation, or manufacturing action involved), the type of defect and may immediately dispose of a rejected glove or reroute it to be used for a non-medical purpose. In some embodiments, this may be accomplished by utilizing a trigger, wherein if the detection system detects a defect, then the trigger may be activated. In some embodiments activating the trigger may effect an accept-or-reject sorting function or algorithm to accept or reject the detected garment based on whether or not that garment is suitable for a specific predetermined purpose. An accept-or-reject sorting function may be preprogrammed with acceptable limits for specific uses such as medical, general, clean room, or the like. These may be based on standards set forth from agencies such as but not limited to the FDA or the ASTM.

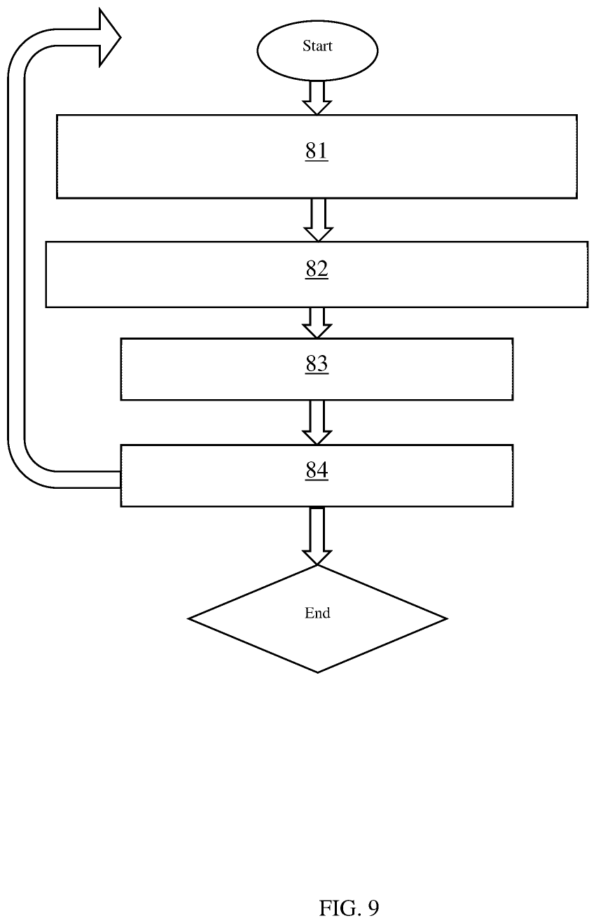

FIG. 9 is an exemplary illustration of a method of elastomeric, protective garment light transmission defect detection in accordance with some embodiments of the technology described herein. In some embodiments, a method of elastomeric, protective garment light transmission defect detection may include but is not limited to the steps of inflating a garment in a negative pressure vacuum chamber, wherein the step of inflating the garment creates an inflated garment (81), emitting a light source through the inflated garment (82), detecting the emitted light source (83), and detecting a garment defect (84). In some embodiments, emitting a light source through the inflated garment may be accomplished by projecting a laser, at least one laser, laser array, LED, LED array, or the like. In some embodiments, detecting light from a light source may be accomplished by detecting light using a variety of different technologies and methodologies including but not limited to detecting with a garment internal camera, a garment external camera, a photosensor array, a reflected light detector, a pass-through light detector, or the like. In one embodiment, detecting light may be accomplished by a garment external camera, a garment internal camera, a photosensor array, a reflected light detector, or a pass-through light detector, or the like. A reflected light detector may be but is not limited to a detector, camera, photosensor, or the like that may measure the light reflected off a garment to detect possible defects. In another embodiment detecting light may accomplished by utilizing a pass-through light detector that may detect light that passes through pinholes, cuts, thickness variabilities, or the like in a garment.

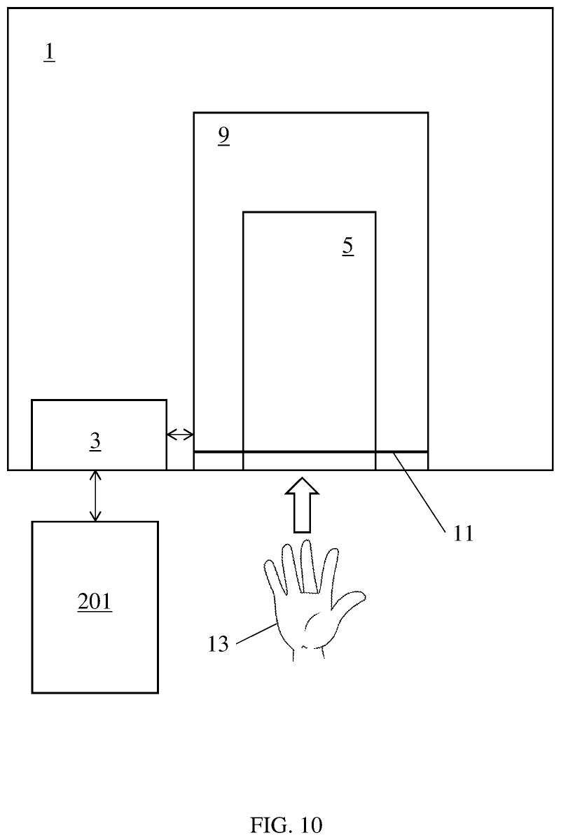

FIG. 10 is an exemplary elastomeric, protective garment pre-containment testing system further illustrating a threshold contaminant detection system data storage system configured to connect to a cloud computing network. In some embodiments an elastomeric, protective garment pre-containment testing system may further include a threshold contaminant detection system data storage system configured to connect to a cloud computing network (201). In some embodiments threshold contaminant detection system data storage system may be similar to a threshold contaminant detection system that may have the ability to send detected contaminant data to a storage system via wireless, wired, or other similar transmission means to an internal or external server connected to the world wide web, the cloud, or the like. In some embodiments the main storage element, that may be an internet or cloud-based network or server, physical server, or the like may have pre-programed predetermined contaminate thresholds that correspond to public health threats. Such a system may be useful in that it can pull and store contaminant data from many different contamination sites to determine if there is a public health threat before it happens. Stored contamination data on a main storage element may be configured to alert for pre-determined public health threats. In some embodiments a pre-determined public health threat may be a threshold limit for certain bacteria, viruses, pathogens, or the like. In other embodiments, an AI system may be used to statistically predict a public health threat.

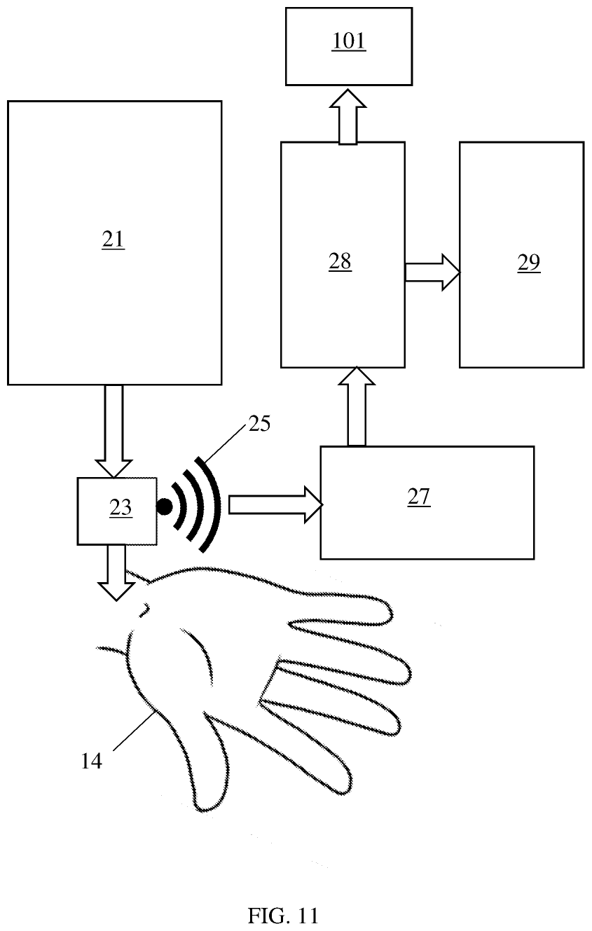

FIG. 11 is an exemplary elastomeric, protective garment tracking system illustrating one embodiment connected to a cloud computing network. In another embodiment, it may be beneficial to connect the donning, doffing, containing, manufacturing, and testing system to the internet, a cloud computing network, or a similar global database, either wirelessly, wired, or cloud based, so that potential detected contaminates may be tracked for public surveillance purposes. This may be beneficial in pinpointing the location and contact group of a particulate contaminate that may pose a threat to public health. In some embodiments, a data storage system may be a data storage system configured to a cloud computing network. In some embodiments a cloud computing network may be a network of elastomeric, protective garment donning, doffing, manufacturing, or containing systems or the like that may be connected through the world wide web, a cloud computing network, internal or external server, or the like. In some embodiments a plurality of elastomeric, protective garment tracking systems may be utilized to create a network of contamination data across a team, facility, city, or the like. In some embodiments the plurality of elastomeric, protective garment tracking systems may be configured to send data, such as contamination data, to a main storage element (101). In some embodiments, the main storage element may be located on an internal or external server, on the internet, or via a cloud storage solution. Stored contamination data on a main storage element may be configured to alert for pre-determined public health threats. In some embodiments a pre-determined public health threat may be a threshold limit for certain bacteria, viruses, pathogens, or the like. In other embodiments, an AI (artificial intelligence) system may be used to statistically predict a public health threat. In other embodiments, a public health threat may be detected once a garment is doffed. In particular embodiments the doffing system may be able to detect an abnormality in a garment and upload this information to the main storage element. The main storage element may then be able to compile abnormality, defect, or the like data and alert the manufacturer of a possible defect in the manufacturing process. In some embodiments, this alert may be automatically and autonomously performed so that it may trigger altering of the garment manufacturing process. In some embodiments, the alert may trigger public health officials.

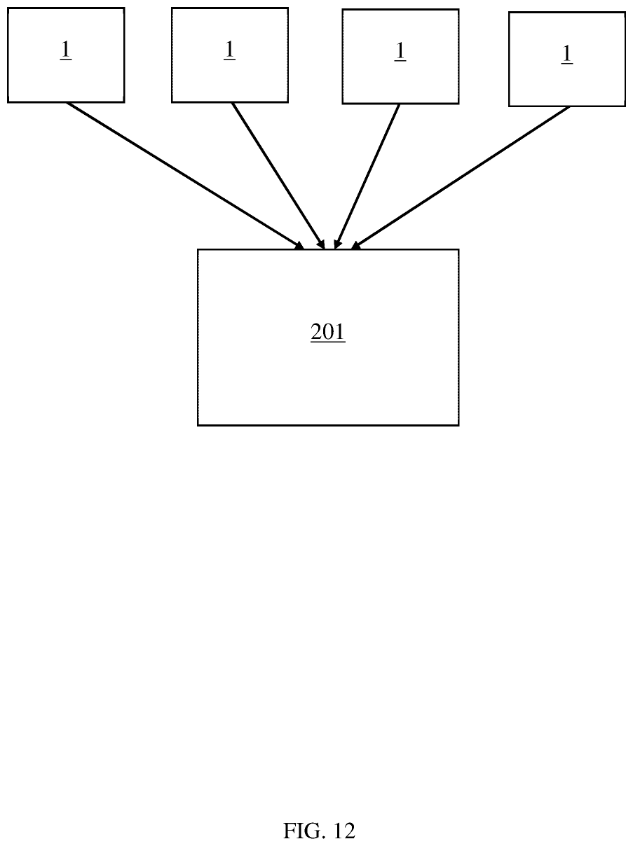

FIG. 12 is an exemplary elastomeric, protective garment pre-containment testing system (1) network configured to connect to a threshold contaminant detection system data storage system configured to connect to a cloud computing network (201). In some embodiments threshold contaminant detection system data storage system may be similar to a threshold contaminant detection system that may have the ability to send detected contaminant data to a storage system via wireless, wired, or other similar transmission means to an internal or external server connected to the world wide web, the cloud, or the like. In some embodiments the main storage element, that may be an internet or cloud-based network or server, physical server, or the like may have pre-programed predetermined contaminate thresholds that correspond to public health threats. Such a system may be useful in that it can pull and store contaminant data from many different contamination sites to determine if there is a public health threat before it happens. Stored contamination data on a main storage element may be configured to alert for pre-determined public health threats. In some embodiments a pre-determined public health threat may be a threshold limit for certain bacteria, viruses, pathogens, or the like. In other embodiments, an AI system may be used to statistically predict a public health threat.

In some embodiments, it may be beneficial to utilize a light source camera detection system to detect defects and material variabilities of a garment before donning. In one embodiment, the light source camera detection system may include but is not limited to a light source and a detector component. A light source may be but is not limited to at least one laser, at least one LED, or an array of lasers or LEDs. In one embodiment, the detector component may be but is not limited to a garment external camera, a garment internal camera, a photosensor array, a reflected light detector, or a pass-through light detector, or the like. A reflected light detector may be but is not limited to a detector, camera, photosensor, or the like that may measure the light reflected off a garment to detect possible defects. In another embodiment the detector component may be a pass-through light detector that may detect light that passes through pinholes, cuts, thickness variabilities, or the like in a garment. The photosensors may utilize a variety of sensors in combination or alone, such as a photoresistor, photo capacitor, LED sensor, or the like. The detector may also include a computer-controlled processor to process the light inputs received by the light detector. The processor may be able to measure the presence of a pinhole or other defect and may be able to detect variabilities in material thickness. In one embodiment, the processor may be useful in detecting even tiny pinhole defects, which may project a bright point of light from that pinhole. Cameras may be located externally that may detect those relatively brighter escapes of light. Alternatively, cameras may be located within the inflated glove and near the source of light or a laser scan which may detect the bright light or non-reflection of light evidenced by the presence of a pinhole. All methods may help reveal other glove defects, such as variations in thickness and thin or weak areas which show up as differences in opacity or translucency.

In some embodiments, a pinhole in a garment may act as a lens similar to that of a pinhole camera magnifying the light projected through that pinhole, similar defect, or the like. In some embodiments as light is projected into an area behind the lens (in this embodiment the garment prepared for defect detection) any defects present in the garment will allow light to project through and will be enlarged on the light collection surface, such as a detector component. In some embodiments a detector component may be a pass-through light detector that may detect light that passes through pinholes, cuts, thickness variabilities, or the like in a garment or may be a photosensor or the like. In some embodiments, photosensors may utilize a variety of sensors in combination or alone, such as a photoresistor, photo capacitor, LED sensor, or the like. The detector may also include a computer-controlled processor to process the light inputs received by the light detector.

In some embodiments, manufacturing feedback may be provided if defects are detected by a corresponding system. A manufacturing feedback system may provide real-time, or close to real-time (such including in seconds, a minute, or coordinated to be accomplished during the specific activity, operation, or manufacturing action involved) feedback to the manufacturing process to adjust based on defects present in the garments. In some embodiments, the manufacturing feedback may be automatic feedback from a detection system, which may not require user input to alter the manufacturing process. This may be done utilizing a computer-controlled function or an artificial intelligence (AI) algorithm. In some embodiments, the automatic manufacturing feedback may be but is not limited to a command configured to alter the material thickness of the garment, a command configured to alter the mold dip time of the garment, a command configured to alter the material cure time of the garment, or a command configured to alter the material formulation of the garment. These and similar parameters may be altered alone or in combination to provide the correct manufacturing feedback based on the data received from the defect detection system.

In some embodiments, a negative pressure removal system may be utilized to assist the user in no-contact sterilized removal of a garment. This may be accomplished by creating a vacuum chamber within a containment zone where said garment provides one sealing edge of the vacuum chamber. In another embodiment, there may be a sealing cuff configured to seal the garment to the negative pressure vacuum chamber. It may be beneficial that the vacuum chamber be a sterile environment in case there is contamination that needs to be tracked from the garment. This may be accomplished by using UV sterilizers within the vacuum chamber or any other type of sterilization.

In some embodiments, it may be beneficial to provide simultaneous sterilizing while a user dons the garment. This may be accomplished by providing a sterilizing element to the system. In some embodiments, the sterilizing element may be inside the donning chamber or may be a pass-through plane that the user may draw, for example, the gloved user's hand, through the sterilizing element. In some embodiments, the sterilizing element may be but is not limited to a biocidal light, a UV-C light, a far UV-C light, a TiO2, AgNO3, chlorhexidine, or a similar compound spray, mist, or powder element. In some embodiments, a spray element may be used as the sterilizing element and may be accompanied with a biocidal, UV, or the like light to sterilize more efficiently and effectively. In some embodiments the spray element may be a TiO2, AgNO3, chlorhexidine, or a similar compound spray, mist, or powder element, or the like. In some embodiments, the garment may be simultaneously sterilized and donned. This may provide an extra layer of protection against contaminants.

In some embodiments, a glove or other garment may be isolatedly contained by containing the garment within a separate sterile environment to further test and study possible contaminants on the garment. In some embodiments, a sterile environment containment may be where a dirty glove or garment goes into a clean bag and gets sealed for isolated containment for further testing. This process may take place during the glove or garment removal process. In some embodiments, sterile environment containment may be but is not limited to a separate containment bag, which may be a separate containment glove, a fully sealed containment bag, an isolated hazardous material bag, or the like. In some embodiments, the containment glove may be a glove of the same type that is pre-inflated, and when a user goes to remove the contaminated or used glove, inserts the gloved hand into the removal machine with a containment glove inflated, and the contaminated or used user glove may then be isolated in the containment glove limiting the risk of cross-contamination. The same or similar process may be used for the fully sealed containment bag and isolated hazardous material bag. In some embodiments, this system may include a tag system configured to store data related to a contaminated garment with the separate containment bag to ensure proper tracking and containment of contaminated garments. The tag system may be but is not limited to an RFID tag, a printed barcode, Bluetooth transmitter, NFC transmitter, LoRa transmitter, wireless transmitter, or the like that provides an output, said output may be a physical sending of data or the ability to be recognized by a receiving system.

Another embodiment may include a real-time alarm system configured to alarm if a garment defect is detected when a user dons the garment. The system may include a garment don and garment doff alarm that may alert the user of garment defects such as but not limited to contaminants present on, or holes in, the garment. In some embodiments, the don alarm trigger may alarm for a manufacturing defect such as but not limited to a pinhole, tear, or material thickness variability. The don alarm trigger may also alarm for contaminants present after the garment is donned. Similarly, the doff alarm trigger may alarm for a use defect such as a cut in the garment material, which may alert the user of possible contamination to the skin. The doff alarm trigger may also alarm for contaminates located on the exterior of the garment. This may alert the user that the garment needs to be isolated or that the system needs to be sterilized prior to the next use. In some embodiments, this may be done automatically through the use of computer-controlled systems, or in some embodiments, it may be done manually by a user.

Some embodiments may include a garment packaging system. A garment packaging system may include but is not limited to a packaging fixture, a defect detection system, a garment sterilization system, and a repackaging system. In some embodiments, gloves may be packaged or repackaged in a process post-manufacture. In some embodiments, this process may or may not be a part of the manufacturing line. In some embodiments, a garment packaging system may include a garment sterilization system such as a sterilizing element. In some embodiments, the sterilizing element may be but is not limited to a biocidal light, a UV-C light, a far UV-C light, a TiO2, AgNO3, chlorhexidine, or a similar compound spray, mist, or powder element. In some embodiments, a spray element may be used as the sterilizing element and may be accompanied with a biocidal, UV, or the like light to sterilize more efficiently and effectively. In some embodiments, the spray element may be a TiO2, AgNO3, chlorhexidine, or a similar compound spray, mist, or powder element, or the like. In some embodiments, the garment may be simultaneously sterilized and donned. In another embodiment, it may be beneficial to include a garment tracking tag embedding system in order for the garment to be serialized with a barcode or embedded RFID. In some embodiments, the tracking tag embedding system may be but is not limited to a user applying a locator tag to each garment; this process may also be automated. The tracking tag embedding system may also be done during the manufacturing or donning phase of the garment through the use of automated controls, such as but not limited to roll on, mold in, or printed application. In some embodiments, a remote-tracking tag that may be applied by a track tag embedding system may be used to track the location of the garment and its uses. A remote tracking tag may be but is not limited to an RFID tag, a printed barcode, a molded barcode, Bluetooth transmitter, NFC transmitter, LoRa transmitter, wireless transmitter, or the like that may provide an output. Said output may be a physical transmission or electronic sending of data or the ability to be recognized by a receiving system, or otherwise. In some embodiments, the garment packaging system may be a repackaging system and provide repackaging of garments in single-use packaging or bulk use packaging. It may also allow for uniform or predetermined orientation of the garment (e.g., thumbs to the left) and packing in boxes, other containers, or the like.

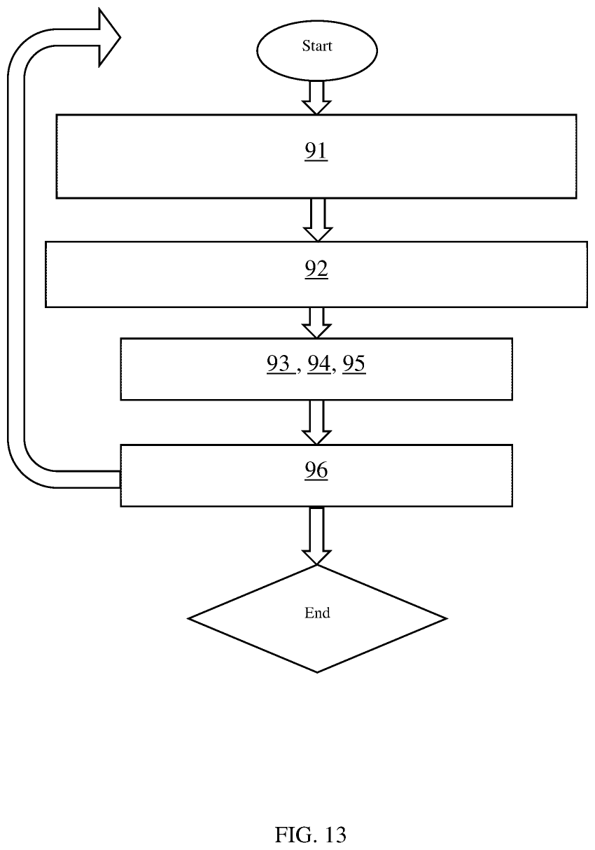

FIG. 13 is an exemplary illustration of a method of elastomeric, protective garment repackaging in accordance with some embodiments of the technology described herein. A method of elastomeric, protective garment repackaging may include but is not limited to the steps removing a garment from a manufacturing environment (91), positioning a garment on a packaging fixture (92), inflating said garment (93), inspecting said garment for defects (94), sterilizing substantially defect-free garments (95), and repackaging a sterilized, substantially defect-free garment (96), or the like. In some embodiments, the step of removing a garment from a manufacturing environment may be accomplished by removing a garment from a manufacturing process such as at the end of the line or from a post-manufacture storage or packaging environment. This may be beneficial for implementing different testing procedures for varying garment certification levels post-manufacture. It may also benefit the speed of the manufacturing process and reduce cost as to not have to retrofit manufacturing lines to also test for garment defects. In another embodiment, positioning the garment on a packaging fixture may be accomplished by manually or automatically placing a garment on a fixture that may aid in repackaging. In some embodiments, this fixture may be a post or hand to accompany a glove. In some embodiments, the packaging fixture may include the capability to include the steps of inflating a garment and inspecting the garment for defects. This may be accomplished by using contained or filtered gas to inflate the glove as to not contaminate it with environmental contaminates, then using defect detection methods to detect for pinholes, irregularities, or the like.

In some embodiments, it may be beneficial to utilize a light source camera detection system to detect defects and material variabilities of a garment before donning. In one embodiment, the light source camera detection system may include but is not limited to a light source and a detector component. A light source may be but is not limited to at least one laser, at least one LED, or an array of lasers or LEDs. In one embodiment, the detector component may be but is not limited to a garment external camera, a garment internal camera, a photosensor array, a reflected light detector, or a pass-through light detector, or the like. A reflected light detector may be but is not limited to a detector, camera, photosensor, or the like that may measure the light reflected off a garment to detect possible defects. In another embodiment, the detector component may be a pass-through light detector that may detect light that passes through pinholes, cuts, thickness variabilities, or the like in a garment. The photosensors may utilize a variety of sensors in combination or alone, such as a photoresistor, photo capacitor, LED sensor, or the like. The detector may also include a computer-controlled processor to process the light inputs received by the light detector. The processor may be able to measure the presence of a pinhole or other defect and may be able to detect variabilities in material thickness. In one embodiment, the processor may be useful in detecting even tiny pinhole defects, which may project a bright point of light from that pinhole. Cameras may be located externally that may detect those relatively brighter escapes of light. Alternatively, cameras may be located within the inflated glove and near the source of light or a laser scan which may detect the bright light or non-reflection of light evidenced by the presence of a pinhole. All methods may help reveal other glove defects, such as variations in thickness and thin or weak areas that show up as differences in opacity or translucency. In some embodiments the packaging fixture may also include a system for embedding a garment tracking tag into the garment. A tracking tag embedding system may be accomplished through the use of manual or automated controls, such as but not limited to roll on, mold in, or printed application. In some embodiments, a remote tracking tag that may be applied by a track tag embedding system may be used to track the location of the garment and its uses. A remote-tracking tag may be but is not limited to an RFID tag, a printed barcode, a molded barcode, Bluetooth transmitter, NFC transmitter, LoRa transmitter, wireless transmitter, or the like that may provide an output. Said output may be a physical transmission or electronic sending of data or the ability to be recognized by a receiving system, or otherwise.

In another embodiment, the step of sterilizing substantially defect-free garments may be accomplished by providing a sterilizing element to the system. In some embodiments, the sterilizing element may be inside the donning chamber or may be a pass-through plane that the user may draw, for example, the gloved user's hand, through the sterilizing element. In some embodiments, the sterilizing element may be but is not limited to a biocidal light, a UV-C light, a far UV-C light, a TiO2, AgNO3, chlorhexidine, or a similar compound spray, mist, or powder element. In some embodiments, a spray element may be used as the sterilizing element and may be accompanied with a biocidal, UV, or the like light to sterilize more efficiently and effectively. In some embodiments, the spray element may be a TiO2, AgNO3, chlorhexidine, or a similar compound spray, mist, or powder element, or the like. In some embodiments, the garment may be simultaneously sterilized and donned. This may provide an extra layer of protection against contaminants.

In some embodiments, the step of repackaging a sterilized, substantially defect-free garment may be accomplished by repackaging a garment in a single garment container or packaging. In other embodiments, repackaging may be accomplished by packaging garments in bulk containers. In some embodiments, a repackaging system may also be capable of orienting garments in a predetermined orientation such as but not limited to glove thumbs to the left, glove palms down, or the like and then package or sterilely contain the garment.

Previously presented definitions of embodiments, together with newly developed donning, doffing, testing, and containment systems and methods, intuitive statements of embodiments from incorporated specifications, are all now incorporated. Clauses for potential assertion include:

1. An elastomeric, protective garment pre-containment testing system comprising:
a garment doffing system;
a threshold contaminant detection system;
a contaminant notification control configured to respond to said threshold contaminant detection system;
a containment system configured to automatically respond to said contaminant notification control, wherein said containment system contains a contaminated garment for further testing, as said garment doffing system removes said contaminated garment from a user.

2. An elastomeric, protective garment pre-containment testing system as described in clause 1 or any other clause wherein said garment doffing system comprises a mechanical garment doffing system.

3. An elastomeric, protective garment pre-containment testing system as described in clause 1 or any other clause wherein said garment doffing system comprises an automatic garment doffing system.

4. An elastomeric, protective garment pre-containment testing system as described in clause 1 or any other clause wherein said garment doffing system comprises a negative pressure garment doffing system.

5. An elastomeric, protective garment pre-containment testing system as described in clause 4 or any other clause wherein said negative pressure garment doffing system comprises a vacuum chamber configured to apply a lower than atmospheric pressure to the outside of said contaminated garment.

6. An elastomeric, protective garment pre-containment testing system as described in clause 1 or any other clause wherein said garment doffing system comprises negative pressure removal system.

7. A pre-containment testing system as described in clause 6 or any other clause wherein said negative pressure removal system comprises creating a vacuum chamber within a containment zone.

8. A pre-containment testing system as described in clause 6 or any other clause wherein said negative pressure removal system comprises a sealing cuff configured to removably seal a garment to a vacuum chamber.

9. An elastomeric, protective garment pre-containment testing system as described in clause 1 or any other clause wherein said threshold contaminant detection system comprises a contaminant scanner.

10. An elastomeric, protective garment pre-containment testing system as described in clause 1 or any other clause wherein said contaminant scanner comprises a spectral scanner.

11. An elastomeric, protective garment pre-containment testing system as described in clause 1 or any other clause wherein said contaminant scanner comprises an immunofluorescence scanner.

12. An elastomeric, protective garment pre-containment testing system as described in clause 1 or any other clause wherein said contaminant notification control comprises a visual signal.

13. An elastomeric, protective garment pre-containment testing system as described in clause 1 or any other clause wherein said contaminant notification control comprises an audible signal.

14. An elastomeric, protective garment pre-containment testing system as described in clause 1 or any other clause wherein said contaminant notification control comprises a sensory signal.

15. An elastomeric, protective garment pre-containment testing system as described in clause 1, or any other clause wherein said a containment system comprises an isolated compartment.

16. An elastomeric, protective garment pre-containment testing system as described in clause 1 or any other clause wherein said a containment system comprises a separable sealable bag.

17. An elastomeric, protective garment pre-containment testing system as described in clause 1 or any other clause wherein said a containment system comprises a separable sterile garment.

18. An elastomeric, protective garment pre-containment testing system as described in clause 1 or any other clause further comprising a threshold contaminant detection system data storage system configured to connect to a cloud computing network.

19. An elastomeric, protective garment pre-containment testing system as described in clause 18 or any other clause wherein said connection to a cloud computing network comprises a connection of a plurality of elastomeric, protective garment pre-containment testing systems.

20. An elastomeric, protective garment pre-containment testing system as described in clause 19 or any other clause wherein said connection of a plurality of elastomeric, protective garment tracking systems comprises a connection of a plurality of elastomeric, protective garment tracking systems configured to send data to a main storage element.

21. An elastomeric, protective garment pre-containment testing system as described in clause 20 or any other clause wherein said main storage element comprises a main storage element configured to alert for pre-determined public health threats.

22. An elastomeric, protective garment pre-containment testing system as described in clause 20 or any other clause wherein said main storage element comprises a cloud storage network.

23. An elastomeric, protective garment pre-containment testing system as described in clause 20 or any other clause wherein said main storage element utilizes collective data for public health purposes.

24. An elastomeric, protective garment tracking system comprising:
a tracking tag embedding system;
a remote tracking tag, wherein said remote tracking tag is attached to a garment by said embedding system;
a remote tracking tag output;
a receiving system, wherein said remote tracking tag output is input to said receiving system;
a data storage system, wherein an input from said receiving system is stored on computer-readable media;
a contaminant locator, wherein if contaminants are present at a location where said garment has been then a tag is added to said computer-readable media.

25. An elastomeric, protective garment tracking system as described in clause 24 or any other clause wherein said remote tracking tag comprises a removable remote tracking tag from said garment.

26. An elastomeric, protective garment tracking system as described in clause 19 or any other clause wherein said removable remote tracking tag comprises a removable remote tracking tag configured to be reused on a clean garment.

27. An elastomeric, protective garment tracking system as described in clause 24 or any other clause wherein said tracking tag embedding system comprises a roll on adhesive remote tagging system.

28. An elastomeric, protective garment tracking system as described in clause 24 or any other clause wherein said tracking tag embedding system comprises an automatic tracking tag embedding system.

29. An elastomeric, protective garment tracking system as described in clause 24 or any other clause wherein said tracking tag embedding system comprises a mold in remote tagging system.

30. An elastomeric, protective garment tracking system as described in clause 24 or any other clause wherein said tracking tag embedding system comprises a printed on remote tagging system.

31. An elastomeric, protective garment tracking system as described in clause 24 or any other clause wherein said a remote tracking tag comprises an RFID enabled remote tracking tag.

32. An elastomeric, protective garment tracking system as described in clause 24 or any other clause wherein said a remote tracking tag comprises a barcode tracking tag.

33. An elastomeric, protective garment tracking system as described in clause 24 or any other clause wherein said a remote tracking tag output comprises an RFID signal.

34. An elastomeric, protective garment tracking system as described in clause 24 or any other clause wherein said a remote tracking tag output comprises scanned barcode data.

35. An elastomeric, protective garment tracking system as described in clause 24 or any other clause wherein said a receiving system comprises an RFID receiver.

36. An elastomeric, protective garment tracking system as described in clause 24 or any other clause wherein said a receiving system comprises a barcode scanner.

37. An elastomeric, protective garment tracking system as described in clause 24 or any other clause wherein said a data storage system comprises a computer.

38. An elastomeric, protective garment tracking system as described in clause 24 or any other clause wherein said a data storage system comprises at least one hard disk.

39. An elastomeric, protective garment tracking system as described in clause 24 or any other clause wherein said a data storage system comprises a network of cloud storage.

40. An elastomeric, protective garment tracking system as described in clause 24 or any other clause wherein said a contaminant locator comprises a room entry scan.

41. An elastomeric, protective garment tracking system as described in clause 24 or any other clause wherein said a contaminant locator comprises a new task scan.

42. An elastomeric, protective garment tracking system as described in clause 24 or any other clause wherein said a tag comprises a location tag.

43. An elastomeric, protective garment tracking system as described in clause 24 or any other clause wherein said a tag comprises a time of contact point tag.

44. An elastomeric, protective garment tracking system as described in clause 24 or any other clause wherein said a tag comprises a time of scan tag.

45. An elastomeric, protective garment tracking system as described in clause 24 or any other clause wherein said a tag comprises a type of contaminant tag.

46. An elastomeric, protective garment tracking system as described in clause 24 or any other clause wherein said data storage system comprises a data storage system configured to connect to a cloud computing network.

47. An elastomeric, protective garment tracking system as described in clause 46 or any other clause wherein said connection to a cloud computing network comprises a connection of a plurality of elastomeric, protective garment tracking systems.

48. An elastomeric, protective garment tracking system as described in clause 47 or any other clause wherein said connection of a plurality of elastomeric, protective garment tracking systems comprises a connection of a plurality of elastomeric, protective garment tracking systems configured to send data to a main storage element.

49. An elastomeric, protective garment tracking system as described in clause 48 or any other clause wherein said main storage element comprises a main storage element configured to alert for pre-determined public health threats.

50. An elastomeric, protective garment tracking system as described in clause 48 or any other clause wherein said main storage element comprises a cloud storage network.

51. An elastomeric, protective garment pre-containment testing system as described in clause 48 or any other clause wherein said main storage element utilizes collective data for public health purposes.

52. A computer-controlled elastomeric, protective garment defect detection system comprising:
a detection system configured to detect defects in garments; and
a trigger, wherein if said detection system detects a defect then said trigger is configured to trigger an accept-or-reject sorting function,
wherein said accept-or-reject sorting function receives data inputs from said detection system and diverts a detected garment according to a preset accept or reject schedule.

53. A computer-controlled elastomeric, protective garment defect detection system as described in clause 52 or any other clause wherein said preset accept or reject schedule comprises acceptable limits for agency standards.

54. A computer-controlled elastomeric, protective garment defect detection system as described in clause 52 or any other clause wherein said detection system configured to detect defects in garments comprises an elastomeric, protective garment light transmission defect detection system.

55. A computer-controlled elastomeric, protective garment defect detection system as described in clause 52 or any other clause wherein said detection system configured to detect defects in garments comprises a computer-controlled vison system.

56. A computer-controlled elastomeric, protective garment defect detection system as described in clause 52 or any other clause further comprising an automatic feedback from said detection system configured to detect defects in garments.

57. A computer-controlled elastomeric, protective garment defect detection system as described in clause 56 or any other clause wherein said automatic feedback comprises an automatic garment material thickness alteration control.

58. A computer-controlled elastomeric, protective garment defect detection system as described in clause 56 or any other clause wherein said automatic feedback comprises an automatic garment mold dip time alteration control.

59. A computer-controlled elastomeric, protective garment defect detection system as described in clause 56 or any other clause wherein said automatic feedback comprises an automatic garment material cure time alteration control.

60. A computer-controlled elastomeric, protective garment defect detection system as described in clause 56 or any other clause wherein said automatic feedback comprises an automatic garment material formulation alteration control.

61. An elastomeric, protective garment light transmission defect detection system as described in clause 52 or any other clause further comprising a computer vision algorithm.

62. An elastomeric, protective garment light transmission defect detection system as described in clause 61 or any other clause wherein said computer vision algorithm comprises an artificial intelligence system configured to learn from detecting light as garment inflation is varied.

63. An elastomeric, protective garment light transmission defect detection system comprising:
a light detection device;
a light source;
a negative pressure garment inflation chamber, wherein said light source shines from a first end of said negative pressure inflation chamber to a second end, wherein said light detection device is positioned at said second end of said negative pressure garment inflation chamber, wherein said light detection device measures light transmission through an inflated garment.

64. An elastomeric, protective garment light transmission defect detection system as described in clause 63 or any other clause wherein said light detection device comprises a garment external camera.

65. An elastomeric, protective garment light transmission defect detection system as described in clause 63 or any other clause wherein said light detection device comprises a garment internal camera.

66. An elastomeric, protective garment light transmission defect detection system as described in clause 63 or any other clause wherein said light detection device comprises a photosensor array.

67. An elastomeric, protective garment light transmission defect detection system as described in clause 63 or any other clause wherein said light detection device comprises a reflected light detector.

68. An elastomeric, protective garment light transmission defect detection system as described in clause 63 or any other clause wherein said light detection device comprises a pass-through light detector.

69. An elastomeric, protective garment light transmission defect detection system as described in clause 63 or any other clause wherein said light source comprises an LED.

70. An elastomeric, protective garment light transmission defect detection system as described in clause 63 or any other clause wherein said light source comprises an LED array.

71. An elastomeric, protective garment light transmission defect detection system as described in clause 63 or any other clause wherein said light source comprises a laser.

72. An elastomeric, protective garment light transmission defect detection system as described in clause 63 or any other clause wherein said light source comprises a laser array.

73. An elastomeric, protective garment light transmission defect detection system as described in clause 63 or any other clause wherein said first end of said negative pressure inflation chamber comprises a garment opening end.

74. An elastomeric, protective garment light transmission defect detection system as described in clause 63 or any other clause wherein said second end of said negative pressure inflation chamber comprises a garment opening end.

75. An elastomeric, protective garment donning, doffing, manufacturing, testing, packaging, or containing system as described in clause 1, 24, 52, 63, 174, or any other clause further comprising a light source camera detection system.

76. An elastomeric, protective garment donning, doffing, manufacturing, testing, packaging, or containing system as described in clause 1, 24, 52, 63, 174, or any other clause wherein said light source camera detection system comprises at least one light source and at least one light detector component.

77. An elastomeric, protective garment donning, doffing, manufacturing, testing, packaging, or containing system as described in clause 1, 24, 52, 63, 174, or any other clause wherein said at least one light source comprises at least one laser.

78. An elastomeric, protective garment donning, doffing, manufacturing, testing, packaging, or containing system as described in clause 1, 24, 52, 63, 174, or any other clause wherein said light source comprises at least one light-emitting diode.

79. An elastomeric, protective garment donning, doffing, manufacturing, testing, packaging, or containing system as described in clause 1, 24, 52, 63, 174, or any other clause wherein said at least one light detector component comprises a garment external camera.

80. An elastomeric, protective garment donning, doffing, manufacturing, testing, packaging, or containing system as described in clause 1, 24, 52, 63, 174, or any other clause wherein said light detector component comprises a garment internal camera.

81. An elastomeric, protective garment donning, doffing, manufacturing, testing, packaging, or containing system as described in clause 1, 24, 52, 63, 174, or any other clause wherein said light detector component comprises a photosensor array.

82. An elastomeric, protective garment donning, doffing, manufacturing, testing, packaging, or containing system as described in clause 1, 24, 52, 63, 174, or any other clause wherein said light detector component comprises a reflected light detector.

83. An elastomeric, protective garment donning, doffing, manufacturing, testing, packaging, or containing system as described in clause 1, 24, 52, 63, 174, or any other clause wherein said light detector component comprises a pass-through light detector.

84. An elastomeric, protective garment donning, doffing, manufacturing, testing, packaging, or containing system as described in clause 1, 24, 52, 63, 174, or any other clause further comprising a negative pressure removal system.

85. An elastomeric, protective garment donning, doffing, manufacturing, testing, packaging, or containing system as described in clause 1, 24, 52, 63, 174, or any other clause wherein said negative pressure removal system comprises creating a vacuum chamber within a containment zone.

86. An elastomeric, protective garment donning, doffing, manufacturing, testing, packaging, or containing system as described in clause 1, 24, 52, 63, 174, or any other clause wherein said negative pressure removal system comprises a sealing cuff configured to removably seal a garment to a vacuum chamber.

87. An elastomeric, protective garment donning, doffing, manufacturing, testing, packaging, or containing system as described in clause 1, 24, 52, 63, 174, or any other clause further comprising a sterile containment vacuum chamber.

88. An elastomeric, protective garment donning, doffing, manufacturing, testing, packaging, or containing system as described in clause 1, 24, 52, 63, 174, or any other clause further comprising a sterilizing element.

89. An elastomeric, protective garment donning, doffing, manufacturing, testing, packaging, or containing system as described in clause 1, 24, 52, 63, 174, or any other clause wherein sterilizing element comprises a biocidal light.

90. An elastomeric, protective garment donning, doffing, manufacturing, testing, packaging, or containing system as described in clause 1, 24, 52, 63, 174, or any other clause wherein biocidal light comprises a UV-C light.

91. An elastomeric, protective garment donning, doffing, manufacturing, testing, packaging, or containing system as described in clause 1, 24, 52, 63, 174, or any other clause wherein biocidal light comprises a far UV-C light.

92. An elastomeric, protective garment donning, doffing, manufacturing, testing, packaging, or containing system as described in clause 1, 24, 52, 63, 174, or any other clause wherein sterilizing element comprises a spray element.

93. An elastomeric, protective garment donning, doffing, manufacturing, testing, packaging, or containing system as described in clause 1, 24, 52, 63, 174, or any other clause further comprising sterile environment containment.

94. An elastomeric, protective garment donning, doffing, manufacturing, testing, packaging, or containing system as described in clause 1, 24, 52, 63, 174, or any other clause wherein sterile environment containment comprises a separate garment containment bag.

95. An elastomeric, protective garment donning, doffing, manufacturing, testing, packaging, or containing system as described in clause 1, 24, 52, 63, 174, or any other clause wherein said separate containment bag comprises a containment glove configured around a contaminated glove as said contaminated glove is removed.

96. An elastomeric, protective garment donning, doffing, manufacturing, testing, packaging, or containing system as described in clause 1, 24, 52, 63, 174, or any other clause wherein said separate containment bag comprises a fully sealed containment bag is configured to fully seal around a contaminated garment upon removal.

97. An elastomeric, protective garment donning, doffing, manufacturing, testing, packaging, or containing system as described in clause 1, 24, 52, 63, 174, or any other clause wherein said separate containment bag comprises an isolated hazardous material bag is configured to fully seal around a contaminated garment upon removal.

98. An elastomeric, protective garment donning, doffing, manufacturing, testing, packaging, or containing system as described in clause 1, 24, 52, 63, 174, or any other clause wherein said separate containment bag comprises a tag system configured to contain data related to a contaminated garment.

99. An elastomeric, protective garment donning, doffing, manufacturing, testing, packaging, or containing system as described in clause 1, 24, 52, 63, 174, or any other clause further comprising a real-time alarm.

100. An elastomeric, protective garment donning, doffing, manufacturing, testing, packaging, or containing system as described in clause 1, 24, 52, 63, 174, or any other clause wherein said real-time alarm comprises a garment don alarm is configured to alarm when a don alarm trigger is detected for a donned garment.

101. An elastomeric, protective garment donning, doffing, manufacturing, testing, packaging, or containing system as described in clause 1, 24, 52, 63, 174, or any other clause wherein said real-time alarm comprises a garment doff alarm configured to alarm when a doff alarm trigger is detected for a doffed garment.

102. An elastomeric, protective garment donning, doffing, manufacturing, testing, packaging, or containing system as described in clause 1, 24, 52, 63, 174, or any other clause wherein said don threshold trigger comprises a manufacturing defect trigger.

103. An elastomeric, protective garment donning, doffing, manufacturing, testing, packaging, or containing system as described in clause 1, 24, 52, 63, 174, or any other clause wherein said don threshold trigger comprises a contaminant trigger.

104. An elastomeric, protective garment donning, doffing, manufacturing, testing, packaging, or containing system as described in clause 1, 24, 52, 63, 174, or any other clause wherein said doff threshold trigger comprises a use defect trigger.

105. An elastomeric, protective garment donning, doffing, manufacturing, testing, packaging, or containing system as described in clause 1, 24, 52, 63, 174, or any other clause wherein said doff threshold trigger comprises a contaminant trigger.

106. A method of elastomeric, protective garment pre-containment testing comprising the steps of:
removing a garment automatically from a user;
testing said garment for contaminants;
automatically detecting contaminants;
triggering a response for detected contaminants; and
automatically containing a contaminated garment.

107. A method of elastomeric, protective garment pre-containment testing as described in clause 106 or any other clause wherein said step of removing a garment automatically from a user comprises removing a garment automatically from a user utilizing a garment doffing system.

108. A method of elastomeric, protective garment pre-containment testing as described in clause 106 or any other clause wherein said step of removing a garment automatically from a user comprises removing a garment automatically from a user utilizing a negative pressure garment doffing system.

109. A method of elastomeric, protective garment pre-containment testing as described in clause 106 or any other clause wherein said step of testing said garment for contaminants comprises passing said garment through a threshold contaminant testing system.

110. A method of elastomeric, protective garment pre-containment testing as described in clause 106 or any other clause wherein said step of triggering a response for detected contaminants comprises notifying a contaminant notification control.

111. A method of elastomeric, protective garment pre-containment testing as described in clause 106 or any other clause wherein said step of automatically containing a contaminated garment comprises containing said garment in a containment system.

112. A method of elastomeric, protective garment pre-containment testing as described in clause 106 or any other clause further comprising the step of storing threshold contaminant detection system data on a main storage element configured to connect to a cloud computing network.

113. A method of elastomeric, protective garment pre-containment testing as described in clause 106 or any other clause further comprising the step of connecting a plurality of elastomeric, protective garment pre-containment testing system to a main storage element configured to connect to a cloud computing network.

114. A method of elastomeric, protective garment pre-containment testing as described in clause 106 or any other clause wherein said step of storing threshold contaminant detection system data on a main storage element configured to connect to a cloud computing network comprises storing threshold contaminant detection system data on a cloud storage network.

115. A method of elastomeric, protective garment pre-containment testing as described in clause 106 or any other clause further comprising the step of alerting for pre-determined public health threats.

116. A method of elastomeric, protective garment pre-containment testing as described in clause 106 or any other clause further comprising the step of utilizing collective threshold contaminant detection system data for public health purposes.

117. A method of elastomeric, protective garment tracking comprising the steps of:
embedding a remote tracking tag to a garment;
receiving an output from said remote tracking tag to a receiving system;
storing data input into said receiving system in a data storage system;
analyzing said data; and
tracking the point of contamination based on analyzed data.

118. A method of elastomeric, protective garment tracking as described in clause 117 or any other clause wherein said step of embedding a remote tracking tag to a garment comprises rolling on an adhesive remote tracking tag.

119. A method of elastomeric, protective garment tracking as described in clause 117 or any other clause wherein said step of embedding a remote tracking tag to a garment comprises automatically embedding a remote tracking tag.

120. A method of elastomeric, protective garment tracking as described in clause 117 or any other clause wherein said step of embedding a remote tracking tag to a garment comprises molding said remote tracking tag into said garment.

121. A method of elastomeric, protective garment tracking as described in clause 117 or any other clause wherein said step of embedding a remote tracking tag to a garment comprises printing said remote tracking tag on to said garment.

122. A method of elastomeric, protective garment tracking as described in clause 117 or any other clause wherein said step of receiving an output from said remote tracking tag to a receiving system comprises receiving an RFID signal.

123. A method of elastomeric, protective garment tracking as described in clause 117 or any other clause wherein said step of receiving an output from said remote tracking tag to a receiving system comprises scanning a barcode.

124. A method of elastomeric, protective garment tracking as described in clause 117 or any other clause wherein said step of storing data input into said receiving system in a data storage system comprises storing data on a system capable of storing computer-readable media.

125. A method of elastomeric, protective garment tracking as described in clause 117 or any other clause wherein said step of storing data input into said receiving system in a data storage system comprises storing data on cloud storage.

126: A method of computer-controlled elastomeric, protective garment defect detection comprising the steps of:
detecting defects in garments;
triggering when said defects in garments are detected;
accepting garments that meet a predetermined quality threshold; and
rejecting garments that fail to meet a predetermined quality threshold.

127. A method of computer-controlled elastomeric, protective garment defect detection as described in clause 126 or any other clause further comprising a step of detecting feedback from a detection system configured to automatically alter a manufacturing process.

128. A method of computer-controlled elastomeric, protective garment defect detection as described in clause 102 or any other clause wherein said step of detecting feedback from a detection system configured to automatically alter a manufacturing process comprises altering the material thickness of a manufactured garment.

129. A method of computer-controlled elastomeric, protective garment defect detection as described in clause 102 or any other clause wherein said step of detecting feedback from a detection system configured to automatically alter a manufacturing process comprises altering the mold dip time of a manufactured garment.

130. A method of computer-controlled elastomeric, protective garment defect detection as described in clause 102 or any other clause wherein said step of detecting feedback from a detection system configured to automatically alter a manufacturing process comprises altering the material cure time of a manufactured garment.

131. A method of computer-controlled elastomeric, protective garment defect detection as described in clause 102 or any other clause wherein said step of detecting feedback from a detection system configured to automatically alter a manufacturing process comprises altering the material formulation of a manufactured garment.

132. A method of computer-controlled elastomeric, protective garment defect detection as described in clause 102 or any other clause further comprising a computer vision system.

133. A method of computer-controlled elastomeric, protective garment defect detection as described in clause 113 or any other clause wherein said computer vision system comprises an artificial intelligence system configured to analyze a garment based on light analysis.

134. A method of elastomeric, protective garment tracking as described in clause 117 or any other clause further comprising utilizing collective defect detection system data for public health purposes.

135. A method of elastomeric, protective garment light transmission defect detection comprising the steps of:
inflating a garment in a negative pressure vacuum chamber, wherein said step of inflating said garment creates an inflated garment;
emitting a light source through said inflated garment;
detecting said emitted light source; and
detecting a garment defect.

136. A method of elastomeric, protective garment light transmission defect detection as described in clause 135 or any other clause wherein said step of emitting a light source through said inflated garment comprises projecting at least one laser.

137. A method of elastomeric, protective garment light transmission defect detection as described in clause 135 or any other clause wherein said step of emitting a light source through said inflated garment comprises projecting at least one light-emitting diode.

138. A method of elastomeric, protective garment light transmission defect detection as described in clause 135 or any other clause wherein detecting said emitted light source comprises detecting light with a garment external camera.

139. A method of elastomeric, protective garment light transmission defect detection as described in clause 135 or any other clause wherein said step of detecting light from a light source comprises detecting light with a garment internal camera.

140. A method of elastomeric, protective garment light transmission defect detection as described in clause 135 or any other clause wherein detecting said emitted light source comprises detecting light with a photosensor array.

141. A method of elastomeric, protective garment light transmission defect detection as described in clause 135 or any other clause wherein detecting said emitted light source comprises detecting light with a reflected light detector.

142. A method of elastomeric, protective garment light transmission defect detection as described in clause 135 or any other clause wherein detecting said emitted light source comprises detecting light a pass-through light detector.

143. A method of elastomeric, protective garment donning, doffing, manufacturing, testing, packaging or containing as described in clause 106, 117, 126, 135, 187, or any other clause further comprising the step of projecting a light source.

144. A method of elastomeric, protective garment donning, doffing, manufacturing, testing, packaging or containing as described in clause 106, 117, 126, 135, 187, or any other clause wherein said step of projecting a light source comprises projecting at least one laser.

145. A method of elastomeric, protective garment donning, doffing, manufacturing, testing, packaging or containing as described in clause 106, 117, 126, 135, 187, or any other clause wherein said light projecting a light source comprises projecting at least one light-emitting diode.

146. A method of elastomeric, protective garment donning, doffing, manufacturing, testing, packaging or containing as described in clause 106, 117, 126, 135, 187, or any other clause further comprising a step of detecting light from a light source.

147. A method of elastomeric, protective garment donning, doffing, manufacturing, testing, packaging or containing as described in clause 106, 117, 126, 135, 187, or any other clause wherein said step of detecting light from a light source comprises detecting light with a garment external camera.

148. A method of elastomeric, protective garment donning, doffing, manufacturing, testing, packaging or containing as described in clause 106, 117, 126, 135, 187, or any other clause wherein said step of detecting light from a light source comprises detecting light with a garment internal camera.

149. A method of elastomeric, protective garment donning, doffing, manufacturing, testing, packaging or containing as described in clause 106, 117, 126, 135, 187, or any other clause wherein said step of detecting light from a light source comprises detecting light with a photosensor array.

150. A method of elastomeric, protective garment donning, doffing, manufacturing, testing, packaging or containing as described in clause 106, 117, 126, 135, 187, or any other clause wherein said step of detecting light from a light source comprises detecting light with a reflected light detector.

151. A method of elastomeric, protective garment donning, doffing, manufacturing, testing, packaging or containing as described in clause 106, 117, 126, 135, 187, or any other clause wherein said step of detecting light from a light source comprises detecting light a pass-through light detector.

152. A method of elastomeric, protective garment donning, doffing, manufacturing, testing, packaging or containing as described in clause 106, 117, 126, 135, 187, or any other clause further comprising a step of removing a garment with a negative pressure removal chamber.

153. A method of elastomeric, protective garment donning, doffing, manufacturing, testing, packaging or containing as described in clause 106, 117, 126, 135, 187, or any other clause wherein the step of removing a garment with a negative pressure removal chamber comprises creating a vacuum chamber within a containment zone.

154. A method of elastomeric, protective garment donning, doffing, manufacturing, testing, packaging or containing as described in clause 106, 117, 126, 135, 187, or any other clause wherein the step of removing a garment with a negative pressure removal chamber comprises sealing a cuff of a garment to an opening of a vacuum chamber.

155. A method of elastomeric, protective garment donning, doffing, manufacturing, testing, packaging or containing as described in clause 106, 117, 126, 135, 187, or any other clause wherein the step of removing a garment with a negative pressure removal chamber comprises sterilizing a vacuum chamber.

156. A method of elastomeric, protective garment donning, doffing, manufacturing, testing, packaging or containing as described in clause 106, 117, 126, 135, 187, or any other clause further comprising a step of simultaneously sterilizing a garment and donning said garment.

157. A method of elastomeric, protective garment donning, doffing, manufacturing, testing, packaging or containing as described in clause 106, 117, 126, 135, 187, or any other clause wherein said step of simultaneously sterilizing a garment comprises shining a biocidal light on said garment.

158. A method of elastomeric, protective garment donning, doffing, manufacturing, testing, packaging or containing as described in clause 106, 117, 126, 135, 187, or any other clause wherein said step of shining a biocidal light on said garment comprises shining a UV-C light.

159. A method of elastomeric, protective garment donning, doffing, manufacturing, testing, packaging or containing as described in clause 106, 117, 126, 135, 187, or any other clause wherein said step of shining a biocidal light on said garment comprises shining a far UV-C light.

160. A method of elastomeric, protective garment donning, doffing, manufacturing, testing, packaging or containing as described in clause 106, 117, 126, 135, 187, or any other clause wherein said step of simultaneously sterilizing a garment comprises spraying a garment with TiO2, AgNO3, chlorhexidine or other abiotic.

161. A method of elastomeric, protective garment donning, doffing, manufacturing, testing, packaging or containing as described in clause 106, 117, 126, 135, 187, or any other clause further comprising a step of containing a garment in a sterile environment.

162. A method of elastomeric, protective garment donning, doffing, manufacturing, testing, packaging or containing as described in clause 106, 117, 126, 135, 187, or any other clause wherein said step of containing a garment in a sterile environment comprises containing said garment in a separate containment bag.

163. A method of elastomeric, protective garment donning, doffing, manufacturing, testing, packaging or containing as described in clause 106, 117, 126, 135, 187, or any other clause wherein said step of containing a garment in a sterile environment comprises containing said garment in a second sterile garment applied around said garment.

164. A method of elastomeric, protective garment donning, doffing, manufacturing, testing, packaging or containing as described in clause 106, 117, 126, 135, 187, or any other clause wherein said step of containing a garment in a sterile environment comprises containing said garment in a fully sealed containment bag, or any other clause wherein said fully sealed containment bag is configured to fully seal around a contaminated garment upon removal.

165. A method of elastomeric, protective garment donning, doffing, manufacturing, testing, packaging or containing as described in clause 106, 117, 126, 135, 187, or any other clause wherein said step of containing a garment in a sterile environment comprises containing said garment in an isolated hazardous material bag, or any other clause wherein said isolated hazardous material bag is configured to fully seal around a contaminated garment upon removal.

166. A method of elastomeric, protective garment donning, doffing, manufacturing, testing, packaging or containing as described in clause 106, 117, 126, 135, 187, or any other clause wherein said step of or any other clause wherein said step of containing a garment in a sterile environment comprises tagging said garment with a tag system configured to store data related to said garment.

167. A method of elastomeric, protective garment donning, doffing, manufacturing, testing, packaging or containing as described in clause 106, 117, 126, 135, 187, or any other clause further comprising a step of alarming in real-time for garment defects.

168. A method of elastomeric, protective garment donning, doffing, manufacturing, testing, packaging or containing as described in clause 106, 117, 126, 135, 187, or any other clause wherein said step of alarming in real-time for garment defects comprises detecting when a garment is donned.

169. A method of elastomeric, protective garment donning, doffing, manufacturing, testing, packaging or containing as described in clause 106, 117, 126, 135, 187, or any other clause wherein said step of alarming in real-time for garment defects comprises detecting when a garment is doffed.

170. A method of elastomeric, protective garment donning, doffing, manufacturing, testing, packaging or containing as described in clause 106, 117, 126, 135, 187, or any other clause wherein said step of detecting when a garment is donned comprises detecting a manufacturing defect.

171. A method of elastomeric, protective garment donning, doffing, manufacturing, testing, packaging or containing as described in clause 106, 117, 126, 135, 187, or any other clause wherein said step of detecting when a garment is donned comprises detecting a contaminant.

172. A method of elastomeric, protective garment donning, doffing, manufacturing, testing, packaging or containing as described in clause 106, 117, 126, 135, 187, or any other clause wherein said step of detecting when a garment is doffed comprises detecting a use defect.

173. A method of elastomeric, protective garment donning, doffing, manufacturing, testing, packaging or containing as described in clause 106, 117, 126, 135, 187, or any other clause wherein said step of detecting when a garment is doffed comprises detecting a contaminant.

174. An elastomeric, protective garment packaging system comprising:
a packaging fixture;
a defect detection system;
a garment sterilization system; and
a garment packaging system.

175. An elastomeric, protective garment packaging system as described in clause 174 or any other clause wherein said packaging fixture comprises a packaging fixture for defect detection.

176. An elastomeric, protective garment packaging system as described in clause 174 or any other clause wherein said packaging fixture comprises a packaging fixture for garment sterilization.

177. An elastomeric, protective garment packaging system as described in clause 174 or any other clause wherein said packaging fixture comprises a post manufacturing packaging fixture.

178. An elastomeric, protective garment packaging system as described in clause 174 or any other clause wherein said a garment packaging system comprises a single garment repackaging system.

179. An elastomeric, protective garment packaging system as described in clause 174 or any other clause wherein said a garment packaging system comprises a bulk garment repackaging system.

180. An elastomeric, protective garment donning, doffing, manufacturing, testing, packaging, or containing system as described in clause 1, 24, 52, 63, 174, or any other clause further comprising a garment packaging system.

181. An elastomeric, protective garment donning, doffing, manufacturing, testing, or containing system as described in clause 1, 24, 52, 63, 174, or any other clause wherein said garment packaging system comprises a packaging fixture.

182. An elastomeric, protective garment donning, doffing, manufacturing, testing, or containing system as described in clause 1, 24, 52, 63, 174, or any other clause wherein said packaging fixture comprises a packaging fixture for defect detection.

183. An elastomeric, protective garment donning, doffing, manufacturing, testing, or containing system as described in clause 1, 24, 52, 63, 174, or any other clause wherein said packaging fixture comprises a packaging fixture for garment sterilization.

184. An elastomeric, protective garment donning, doffing, manufacturing, testing, or containing system as described in clause 1, 24, 52, 63, 174, or any other clause wherein said packaging fixture comprises a post manufacturing packaging fixture.

185. An elastomeric, protective garment donning, doffing, manufacturing, testing, or containing system as described in clause 1, 24, 52, 63, 174, or any other clause wherein said a garment packaging system comprises a single garment repackaging system.

186. An elastomeric, protective garment donning, doffing, manufacturing, testing, or containing system as described in clause 1, 24, 52, 63, 174, or any other clause wherein said a garment packaging system comprises a bulk garment repackaging system.

187. A method of elastomeric, protective garment repackaging comprising the steps of:
removing a garment from a manufacturing environment;
positioning said garment on a packaging fixture;
inflating said garment;
inspecting said garment for defects;
sterilizing substantially defect-free garments; and
repackaging a sterilized, substantially defect-free garment.

188. A method of elastomeric, protective garment repackaging as described in clause 187 or any other clause wherein said step of removing a garment from a manufacturing environment comprises removing a garment from a single garment location.

189. A method of elastomeric, protective garment repackaging as described in clause 187 or any other clause wherein said step of removing a garment from a manufacturing environment comprises removing a garment from a bulk garment location.

190. A method of elastomeric, protective garment repackaging as described in clause 187 or any other clause wherein said step of removing a garment from a manufacturing environment comprises detecting defects in a garment.

191. A method of elastomeric, protective garment repackaging as described in clause 187 or any other clause wherein said step of inspecting said garment for defects in a garment comprises inflating the garment to detect for defects.

192. A method of elastomeric, protective garment repackaging as described in clause 187 or any other clause wherein said step of sterilizing substantially defect-free garments comprises spraying a garment with TiO2, AgNO3, chlorhexidine or other abiotic.

193. A method of elastomeric, protective garment repackaging as described in clause 187 or any other clause wherein said step of sterilizing substantially defect-free garments comprises shining a biocidal light on said garment.

194. A method of elastomeric, protective garment repackaging as described in clause 193 or any other clause wherein said step of shining a biocidal light on said garment comprises shining a far UV-C light.

195. A method of elastomeric, protective garment repackaging as described in clause 193 or any other clause wherein said step of shining a biocidal light on said garment comprises shining a UV-C light.

196. A method of elastomeric, protective garment repackaging as described in clause 187 or any other clause wherein said step of repackaging a sterilized, substantially defect-free garment comprises orienting a garment in a predetermined orientation to optimized packaging.

197. A method of elastomeric, protective garment repackaging as described in clause 187 or any other clause wherein said step of repackaging a sterilized, substantially defect-free garment comprises repackaging in a single garment container.

198. A method of elastomeric, protective garment repackaging as described in clause 187 or any other clause wherein said step of repackaging a sterilized, substantially defect-free garment comprises repackaging in a bulk garment container.

199. A method of elastomeric, protective garment donning, doffing, manufacturing, testing, packaging or containing as described in clause 106, 117, 126, 135, 187, 187, or any other clause further comprising a step of garment packaging.

200. A method of elastomeric, protective garment donning, doffing, manufacturing, testing, packaging or containing as described in clause 106, 117, 126, 135, 187, 187, or any other clause wherein said step of garment packaging comprises removing a garment from a single garment location.

201. A method of elastomeric, protective garment donning, doffing, manufacturing, testing, packaging or containing as described in clause 106, 117, 126, 135, 187, or any other clause wherein said step of garment packaging comprises removing a garment from a bulk garment location.

202. A method of elastomeric, protective garment donning, doffing, manufacturing, testing, packaging or containing as described in clause 106, 117, 126, 135, 187, or any other clause wherein said step of garment packaging comprises detecting defects in a garment.

203. A method of elastomeric, protective garment donning, doffing, manufacturing, testing, packaging or containing as described in clause 106, 117, 126, 135, 187, or any other clause wherein said step of detecting defects in a garment comprises inflating the garment to detect for defects.

204. A method of elastomeric, protective garment donning, doffing, manufacturing, testing, packaging or containing as described in clause 106, 117, 126, 135, 187, or any other clause wherein said step of garment packaging comprises sterilizing a garment.

205. A method of elastomeric, protective garment donning, doffing, manufacturing, testing, packaging or containing as described in clause 106, 117, 126, 135, 187, or any other clause wherein said step of garment packaging comprises orienting a garment in a predetermined orientation to optimized packaging.

As can be easily understood from the foregoing, the basic concepts of the various embodiments of the present invention(s) may be embodied in a variety of ways. It involves both systems and methods to don, doff, contain, manufacture, test, and analyze gloves and other garments, including techniques as well as devices to accomplish the appropriate donning, doffing, containing, and testing of gloves and other similar garments. In this application, the donning, doffing, containing, and testing of gloves and other similar garments techniques are disclosed as part of the results shown to be achieved by the various devices described and as steps that are inherent to utilization. They are simply the natural result of utilizing the devices as intended and described. In addition, while some devices are disclosed, it should be understood that these not only accomplish certain methods but also may be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the various embodiments of the invention(s) and may not explicitly show how each feature or element can actually be representative of a broader function or of a great variety of alternative or equivalent elements. As one example, terms of degree, terms of approximation, and/or relative terms may be used. These may include terms such as the words: substantially, about, only, and the like. These words and types of words are to be understood in a dictionary sense as terms that encompass an ample or considerable amount, quantity, size, etc. as well as terms that encompass largely but not wholly that which is specified. Further, for this application, if or when used, terms of degree, terms of approximation, and/or relative terms should be understood as also encompassing more precise and even quantitative values that include various levels of precision and the possibility of claims that address a number of quantitative options and alternatives.

For example, to the extent ultimately used, the existence or non-existence of a substance or condition in a particular input, output, or at a particular stage can be specified as substantially only x or substantially free of x, as a value of about x, or such other similar language. Using percentage values as one example, these types of terms should be understood as encompassing the options of percentage values that include 99.5%, 99%, 97%, 95%, 92% or even 90% of the specified value or relative condition; correspondingly for values at the other end of the spectrum (e.g., substantially free of x, these should be understood as encompassing the options of percentage values that include not more than 0.5%, 1%, 3%, 5%, 8% or even 10% of the specified value or relative condition, all whether by volume or by weight as either may be specified). For example, using percentage values as one example, for the defect detection product to be substantially only the desired product, it should be understood that embodiments of the invention may encompass the option of percentage values that include 99.5%, 99%, 97%, 95%, 92% or even 90% of the output being the desired substantially defect-free product. Correspondingly for values at the other end of the spectrum (e.g., substantially free of defects, embodiments of the invention should be understood as encompassing the options of percentage values that include not more than 0.5%, 1%, 3%, 5%, 8% or even 10% as defects whether by volume, by weight, or by count. In context, these should be understood by a person of ordinary skill as being disclosed and included whether in an absolute value sense or in valuing one set of or substance as compared to the value of a second set of or substance. Again, these are implicitly included in this disclosure and should (and, it is believed, would) be understood to a person having ordinary skill in the art of this field. Where the application is described in device-oriented terminology, each element of the device implicitly performs a function. Apparatus claims may not only be included for the device described, but also method or process claims may be included to address the functions of the embodiments and that each element performs. Neither the description nor the terminology is intended to limit the scope of the claims that will be included in any subsequent patent application.

It should also be understood that a variety of changes may be made without departing from the essence of the various embodiments of the invention(s). Such changes are also implicitly included in the description. They still fall within the scope of the various embodiments of the invention(s). A broad disclosure encompassing the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of embodiments of the invention(s) both independently and as an overall system.

Further, each of the various elements of the embodiments of the invention(s) and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the various embodiments of the invention(s), the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which embodiments of the invention(s) is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “detection system” should be understood to encompass disclosure of the act of “detecting”—whether explicitly discussed or not—and, conversely, were there effectively disclosure of the act of “detecting”, such a disclosure should be understood to encompass disclosure of a “detector” and even a “means for detecting.” Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function. As other non-limiting examples, it should be understood that claim elements can also be expressed as any of: components that are configured to, or configured and arranged to, achieve a particular result, use, purpose, situation, function, or operation, or as components that are capable of achieving a particular result, use, purpose, situation, function, or operation. All should be understood as within the scope of this disclosure and written description.

Any patents, publications, or other references mentioned in this application for patent are hereby incorporated by reference. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference.

Finally, all references listed in the list of References To Be Incorporated By Reference or other information statement filed with the application are hereby appended and hereby incorporated by reference, however, as to each of the above, to the extent that such information or statements incorporated by reference might be considered inconsistent with the patenting of this/these invention(s) such statements are expressly not to be considered as made by the applicant(s).

### REFERENCES TO BE INCORPORATED BY REFERENCE

I. U.S. Patents

| | Kind | | | Number |
|---|---|---|---|---|
| U.S. Pat. No. | Code | Date Issued | Patentee | of Pages |
| 1,938,685 | | Dec. 12, 1993 | H. E. Breulus et al. | 3 |
| 3,067,001 | | Dec. 4, 1962 | D. W. McCollum | 8 |
| 3,481,101 | | Dec. 2, 1969 | Steadman | 7 |
| 4,069,913 | | Jan. 24, 1978 | Harrigan | 6 |
| 4,275,812 | | Jun. 30, 1981 | Poncey et al. | 7 |
| 4,773,532 | | Sep. 27, 1988 | Stephenson | 5 |
| 4,889,266 | | Dec. 26, 1989 | Wright | 16 |
| 4,909,413 | | Mar. 20, 1990 | McCutcheon | 10 |
| 4,915,272 | | Apr. 10, 1990 | Vlock | 14 |
| 4,971,233 | | Nov. 20, 1990 | Keenan | 5 |
| 5,058,785 | | Oct. 22, 1991 | Rich et al. | 9 |
| 5,204,632 | | Apr. 20, 1993 | Leach | 8 |
| 5,456,354 | | Oct. 10, 1995 | Wood | 13 |
| 6,193,117 | B1 | Feb. 27, 2001 | Poschelk | 10 |
| 6,213,360 | B1 | Apr. 10, 2001 | Aluisi | 7 |
| 6,375,034 | B1 | Apr. 23, 2002 | Corbett | 9 |
| 6,435,388 | B1 | Apr. 20, 2002 | Binder et al. | 14 |
| 6,554,168 | B2 | Apr. 29, 2003 | Stobart | 4 |
| 6,589,167 | B1 | Jul. 8, 2003 | Shimomura et al. | 15 |
| 7,331,672 | B2 | Feb. 19, 2008 | Spediacci et al. | 30 |
| 7,537,586 | B2 | May 26, 2009 | Kline et al. | 15 |
| 7,635,067 | B1 | Dec. 22, 2009 | Flynn | 13 |
| 7,712,642 | B2 | May 11, 2010 | Gaines et al | 21 |
| 8,479,918 | B2 | Jul. 9, 2013 | Howard | 17 |
| 9,283,444 | B2 | Mar. 15, 2016 | Cohen et al. | 52 |
| 9,330,497 | B2 | May 3, 2016 | Byrd et al. | 25 |
| 9,925,015 | B2 | Mar. 27, 2018 | Gravlee | 16 |

II. U.S. Patent Application Publications

| Publication | Kind | Publication | | Number |
|---|---|---|---|---|
| Number | Code | Date | Applicant | of pages |
| 2002/0113079 | A1 | Aug. 22, 2002 | Corbett | 16 |
| 2004/0149788 | A1 | Aug. 5, 2004 | Sato | 26 |
| 2006/0010563 | A1 | Jan. 19, 2006 | Michel et al. | 9 |
| 2007/0170213 | A1 | Jul. 26, 2007 | Gaines et al. | 22 |
| 2009/0307825 | A1 | Dec. 17, 2009 | Bhalla | 10 |
| 2010/0147909 | A1 | Jun. 17, 2010 | Kelly et al. | 32 |
| 2010/0263695 | A1 | Oct. 21, 2010 | Hampe | 21 |
| 2011/0186589 | A1 | Aug. 4, 2011 | Lee | 16 |
| 2011/0283439 | A1 | Nov. 24, 2011 | Backhaus et al. | 22 |

III. Non-Patent Literature

Automatic Sterile Glove Donning Device,
www.jamesdysonaward.org/en-AU/2021/project/automatic-sterile-glove-
donning-device/
IGIN, ww.youtube.com/channel/UCDKTUX6AGBQbpV9DuBM97zw

Thus, the applicant(s) should be understood to have support to claim and make claims to embodiments including at least: i) each of the donning, doffing, testing, and containing devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such processes, methods, systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

In addition and as to computer aspects and each aspect amenable to programming or other electronic automation, it should be understood that in characterizing these and all other aspects of the various embodiments of the invention(s)—whether characterized as a device, a capability, an element, or otherwise, because all of these can be implemented via software, hardware, or even firmware structures as set up for a general purpose computer, a programmed chip or chipset, an ASIC, application specific controller, subroutine, or other known programmable or circuit specific structure—it should be understood that all such aspects are at least defined by structures including, as person of ordinary skill in the art would well recognize: hardware circuitry, firmware, programmed application specific components, and even a general purpose computer programmed to accomplish the identified aspect. For such items implemented by programmable features, the applicant(s) should be understood to have support to claim and make a statement of invention to at least: xv) processes performed with the aid of or on a computer, machine, or computing machine as described throughout the above discussion, xvi) a programmable apparatus as described throughout the above discussion, xvii) a computer readable memory encoded with data to direct a computer comprising means or elements which function as described throughout the above discussion, xviii) a computer, machine, or computing machine configured as herein disclosed and described, xix) individual or combined subroutines and programs as herein disclosed and described, xx) a carrier medium carrying computer readable code for control of a computer to carry out separately each and every individual and combined method described herein or in any claim, xxi) a computer program to perform separately each and every individual and combined method disclosed, xxii) a computer program containing all and each combination of means for performing each and every individual and combined step disclosed, xxiii) a storage medium storing each computer program disclosed, xxiv) a signal carrying a computer program disclosed, xxv) a processor executing instructions that act to achieve the steps and activities detailed, xxvi) circuitry configurations (including configurations of transistors, gates, and the like) that act to sequence and/or cause actions as detailed, xxvii) computer readable medium(s) storing instructions to execute the steps and cause activities detailed, xxviii) the related methods disclosed and described, xxix) similar, equivalent, and even implicit variations of each of these systems and methods, xxx) those alternative designs which accomplish each of the functions shown as are disclosed and described, xxxi) those alternative designs and methods which accomplish each of the functions shown as are implicit to accomplish that which is disclosed and described, xxxii) each feature, component, and step shown as separate and independent inventions, and xxxiii) the various combinations of each of the above and of any aspect, all without limiting other aspects in addition.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising,” are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 9 or any other claim” or the like, it could be re-drafted as dependent on claim 1, claim 8, or even claim 11 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the various embodiments of the application, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

What is claimed is:

1. An elastomeric, protective garment pre-containment testing system comprising:
a garment doffing system;
a threshold contaminant detection system;
a contaminant notification control configured to respond to said threshold contaminant detection system;
a containment system configured to automatically respond to said contaminant notification control, wherein said containment system contains a contaminated garment for further testing, as said garment doffing system removes said contaminated garment from a user.

2. An elastomeric, protective garment pre-containment testing system as described in claim 1 further comprising an elastomeric, protective garment tracking system.

3. An elastomeric, protective garment pre-containment testing system as described in claim 2 wherein said elastomeric, protective garment tracking system comprises:
a tracking tag embedding system;
a remote tracking tag, wherein said remote tracking tag is attached to a garment by said embedding system;
a remote tracking tag output;
a receiving system, wherein said remote tracking tag output is input to said receiving system;
a data storage system, wherein an input from said receiving system is stored on computer-readable media;
a contaminant locator, wherein if contaminants are present at a location where said garment has been then a tag is added to said computer-readable media.

4. An elastomeric, protective garment pre-containment testing system as described in claim 1 further comprising a computer-controlled elastomeric, protective garment defect detection system.

5. An elastomeric, protective garment pre-containment testing system as described in claim 4 wherein said a computer-controlled elastomeric, protective garment defect detection system comprises:
a detection system configured to detect defects in garments; and
a trigger, wherein if said detection system detects a defect then said trigger is configured to trigger an accept-or-reject sorting function, wherein said accept-or-reject sorting function receives data inputs from said detection system and diverts a detected garment according to a preset accept or reject schedule.

6. An elastomeric, protective garment pre-containment testing system as described in claim 5 wherein said don threshold trigger comprises a manufacturing defect trigger.

7. An elastomeric, protective garment pre-containment testing system as described in claim 1 further comprising an elastomeric, protective garment light transmission defect detection system.

8. An elastomeric, protective garment pre-containment testing system as described in claim 7 wherein said elastomeric, protective garment light transmission defect detection system comprises:
a light detection device;
a light source;
a negative pressure garment inflation chamber, wherein said light source shines from a first end of said negative pressure inflation chamber to a second end, wherein said light detection device is positioned at said second end of said negative pressure garment inflation chamber, wherein said light detection device measures light transmission through an inflated garment.

9. An elastomeric, protective garment pre-containment testing system as described in claim 1 wherein said garment doffing system comprises an automatic garment doffing system.

10. An elastomeric, protective garment pre-containment testing system as described claim 1 wherein said garment doffing system comprises a negative pressure garment doffing system

11. An elastomeric, protective garment pre-containment testing system as described in claim 10 wherein said negative pressure garment doffing system comprises a vacuum chamber configured to apply a lower than atmospheric pressure to the outside of said contaminated garment.

12. A pre-containment testing system as described in clause 6 or any other clause wherein said negative pressure removal system comprises creating a vacuum chamber within a containment zone.

13. A method of elastomeric, protective garment pre-containment testing comprising the steps of:
removing a garment automatically from a user;
testing said garment for contaminants;
automatically detecting contaminants;
triggering a response for detected contaminants; and
automatically containing a contaminated garment.

14. A method of elastomeric, protective garment pre-containment testing as described in claim 13 wherein said step of removing a garment automatically from a user comprises removing a garment automatically from a user utilizing a negative pressure garment doffing system.

15. A method of elastomeric, protective garment pre-containment testing as described in claim 13 wherein said step of testing said garment for contaminants comprises passing said garment through a threshold contaminant testing system.

16. A method of elastomeric, protective garment pre-containment testing as described in claim 13 wherein said step of triggering a response for detected contaminants comprises notifying a contaminant notification control.

17. A method of elastomeric, protective garment pre-containment testing as described in claim 13 further comprising a method of elastomeric, protective garment tracking.

18. A method of elastomeric, protective garment pre-containment testing as described in claim 17 wherein said method of elastomeric, protective garment tracking comprises the steps of:
embedding a remote tracking tag to a garment;
receiving an output from said remote tracking tag to a receiving system;
storing data input into said receiving system in a data storage system;
analyzing said data; and
tracking the point of contamination based on analyzed data.

19. A method of elastomeric, protective garment pre-containment testing as described in claim 13 further comprising a method of computer-controlled elastomeric, protective garment defect detection.

20. A method of elastomeric, protective garment pre-containment testing as described in claim 19 wherein said a method of computer-controlled elastomeric, protective garment defect detection comprises the steps of:
detecting defects in garments;
triggering when said defects in garments are detected;
accepting garments that meet a predetermined quality threshold; and
rejecting garments that fail to meet a predetermined quality threshold.

21. A method of elastomeric, protective garment pre-containment testing as described in claim 20 further comprising a step of detecting feedback from a detection system configured to automatically alter a manufacturing process.

22. A method of computer-controlled elastomeric, protective garment defect detection as described in claim 20 further comprising a computer vision system.

23. A method of computer-controlled elastomeric, protective garment defect detection as described in clause 119 or any other clause wherein said computer vision system comprises an artificial intelligence system configured to analyze a garment based on light analysis.

24. A method of elastomeric, protective garment pre-containment testing as described in claim 13 further comprising a method of elastomeric, protective garment light transmission defect detection.

25. A method of elastomeric, protective garment pre-containment testing as described in claim 24 wherein said method of elastomeric, protective garment light transmission defect detection comprises the steps of:
inflating a garment in a negative pressure vacuum chamber, wherein said step of inflating said garment creates an inflated garment;
emitting a light source through said inflated garment;
detecting said emitted light source; and
detecting a garment defect.

26. A method of elastomeric, protective garment pre-containment testing as described in claim 13 further comprising a step of detecting a manufacturing defect when a garment is donned.
